(19) United States
(12) Patent Application Publication
(10) Pub. No.: US 2019/0099113 A1
(43) Pub. Date: Apr. 4, 2019

(54) METHOD AND DEVICE FOR DETECTING A FALL

(71) Applicants: Gunther Röder, München (DE); Wolfgang Müller-Adam, Brucköbel (DE)

(72) Inventors: Gunther Röder, München (DE); Wolfgang Müller-Adam, Brucköbel (DE)

(21) Appl. No.: 16/068,496

(22) PCT Filed: Jan. 9, 2017
(86) PCT No.: PCT/EP2017/000021

§ 371 Date: Dec. 11, 2018

(30) Foreign Application Priority Data
Jan. 7, 2016 (DE) 10 2016 000 086

(51) Int. Cl. A61B 5/11; A61B 5/00

(52) U.S. Cl. CPC A61B 5/1117; A61B 5/0024; A61B 5/1122; G16H 80/00; A61B 5/7275; A61B 2562/0219; A61B 5/6807

(57) ABSTRACT

Method for detecting a tilt fall or a risk of a tilt fall, proposed with the following steps: detecting a center of gravity position of a center of gravity of the human which represents an actual center of mass of the human or an approximation to it, detecting a position of a standing basis of the human or an approximation to it, detecting connection information regarding a spatial connection between the center of gravity and the standing basis, comprising the steps detecting an angular position of at least one lower leg of a leg to the associated thigh and the angular position of the same thigh to the torso and calculating a relative position of an ankle or at least a part of a foot or a footwear of the leg in relation to the center of gravity as connection information, wherein the angular positions between the lower leg and thigh as well as between thigh and torso as well as lengths of the lower leg and the thigh as well as a relative position between the hip joint of the leg and the torso are used, and deriving a tilt fall or a risk of a tilt fall from the grade of correctness that the center of gravity and the standing basis are located in direction of the acceleration of gravity to each other. Further, the invention concerns an apparatus for carrying out the method as well as a fall protection garment.

This invention concerns a method for recognizing a risk of a tilt fall or tilt fall of a human in which a human falls while being out of his balance. Further, the invention concerns an apparatus for recognizing a risk of fall as well as a fall protection garment with such an apparatus.

A tilt fall is a kind of fall in which the human gets out of his balance and falls over. In this patent application, it is distinguished from a vertical fall in which the human collapses or slumps by failure of support of the center of gravity, for example by passing out or myasthenia.

It is known in the state of the part to use fall protection garment with impact protection dampers. This has the disadvantage that they are often bulky and reduce the suppleness of the protection garment. Furthermore, they can protect the human only at places at which they are attached to the garment. It is further known to integrate an inflatable airbag which is opened at a fall risk. However, no universally applicable and reliable methods for recognizing a risk of fall or a fall are known by help of which the opening of an airbag can be triggered.

In the state of the part, the US 2014/0276242 A1 is known which discloses to detect the state of balance of human by mapping its center of mass on a standing basis. In this, the standing basis is defined as an area on the ground on which the human supports itself with his feet. For mapping the center of mass on the standing basis, it is proposed to draw a vertical line from the center of mass to the standing basis, wherein the point of intersection of this vertical line with the standing basis represents the state of balance. It is proposed to determine a loss of balance when the point of intersection leaves the standing basis farther than 1 inch. However, the US 2014/0276242 A1 quiet regarding details of measurement and calculation of the relative position as well as setting the vertical direction into a relation.

According to the invention, a method for detecting a tilt fall or a risk of tilt fall with the following steps is proposed:
detecting a center of gravity position of a center of gravity of the human which represents an actual center of mass of the human or an approximation thereto,
detecting a standing basis position of the human or an approximation thereto,
detecting a connection information regarding a spatial connection between the center of gravity and the standing basis, comprising the steps
   detecting an angular position of at least a lower leg of a leg to the corresponding thigh and an angular position of the same thigh to the torso,
   calculating as the connection information a relative position of an ankle or at least a part of a foot or a footwear of the leg relating to the center of gravity, wherein angular positions between the lower leg and the thigh as well as between the thigh and the torso as well as lengths of the lower legs and thighs as well as a relative position between the hip joint of the leg and the center of gravity are used,
deriving a tilt fall or a risk of tilt fall from the grade of correctness that the center of gravity and the standing basis are located relative to each other in direction of an acceleration of the center of gravity.

The connecting information can contain information regarding the form of the standing basis. This information can, for example, be present as points and/or lines in a coordinate system which describe the border or single points of the standing basis, and/or as vectors which represent distances between points of the border of the standing basis. Further, the connecting information contains at least one distance between the center of gravity and a point or a line of the standing basis; alternatively or additionally, it can contain information regarding vectors or equivalent data of which said distance is composed. The connecting information can contain fixed and variable portions as is described in detail below. As a fixed portion, the connection information can comprise a distance or a vector which extends from the hip joint to the center of gravity, and, if applicable, a distance or vector between the ankle and the standing basis.

The distance has a direction in space. In the same space, also the acceleration of the center of gravity has a direction. In order to be able to detect to which amount the center of gravity and the standing basis are located relative to each other regarding the direction of the acceleration of the center of gravity, the direction of the distance relative to the acceleration of the center of gravity or to a coordinate system which the distance has in common with the acceleration of the center of gravity, initially can be. In the latter case, the direction of the acceleration of the center of gravity can be determined in the common coordinate system. Then, in both cases, a direction deviation between the direction of the acceleration of the center of gravity and the direction of the distance can be calculated. This direction deviation can be interpreted as a grade of correctness that the center of gravity and the standing basis are located relative to each other in direction of the acceleration of the center of gravity. This equals to carrying out a projection of the center of gravity into the plane of the standing basis or a projection of at least a part of the border of the standing basis into a plane of the center of gravity. In this, the direction of projection is the direction of the acceleration of the center of gravity. This can be handled mathematically in a simple way if the coordinates of the center of gravity and of the standing basis are related to a coordinate system in which a coordinate axis has the direction of the acceleration of the center of gravity. Then, a projection in direction of the acceleration of the center of gravity can be carried out in a simple way by varying the coordinate of the coordinate axis in direction of the acceleration of the center of gravity.

As the acceleration of the center of gravity can, in principle, vary, in case of such variation and/or regularly, an update calculation of the connection information can be carried out. Also, an update calculation of the connection information can be carried out in case of variations and/or regularly. From the connection information that has been obtained successively in time, courses, velocities and acceleration of the center of gravity in relation to the standing basis can be calculated. In an alternative calculation rule, the coordinate system can also be fixed and the direction of the acceleration of the center of gravity can be calculated again. A projection then has a complicated calculation rule, because the projection does not take place in direction of a coordinate axis. For example, the fixed coordinate system can have the longitudinal direction of the lower leg as a coordinate axis. For each of the legs, an own coordinate system can be provided, respectively.

Optionally, exceeding the detection of only tilt falls which occur in consequence of loss of balance, also detection of vertical falls can be carried out in which a person slumps, initially without losing balance. As vertical falls or mixed forms of vertical falls and tilt falls occur frequently, the detection also of vertical falls or portions of vertical fall is referred for an extensive assessment of the risk of fall or of falls.

It is proposed to determine a vertical fall in case that a velocity or an acceleration of a torso of the human in direction to the plane of the standing basis which is greater than a particularly redetermined threshold value is detected, wherein, particularly, a beginning fall is determined if an exceeding of a beginning vertical fall velocity and/or of a beginning vertical fall acceleration is recognized, and/or a ground fall is recognized if an exceeding of a vertical fall ground velocity and/or a vertical fall ground acceleration is recognized. The vertical fall ground velocity may correspond to an impact force which would cause a falling person to suffer significant pain or injuries. The vertical fall ground acceleration can be an acceleration at which one can assume that an extensive loss of control of the person over his movements has occurred. This can, for example, be an acceleration, at which muscle failure in at least one leg occurs. A beginning of a fall which possibly might still be stopped may for example occur by low blood sugar level and a dull state of mind. As the muscles do not completely fail immediately, lower velocities and fall accelerations occur. An alert can be triggered in order to direct the attention of the person on this situation. A risk of a vertical fall can be receded by evidence for a loss of control. For example, this can be an increased bending of the hip, increased curvature of the sine or very quick holding of the head with the hands. Such evidence can be recognized by detection of the posture of the body. As a consequence of such evidence, the seed of data processing can be increased in order to be able to react faster to an actually occurring vertical fall. As a pure vertical fall takes place significantly faster as a tilt fall, time is noticeably more critical in it. The velocity of the torso can be calculated from determination of connection information between the standing basis and center of gravity at multiple succeeding points in time, wherein differences of position information are divided by their distance in time. The acceleration of the torso can be calculated analogously from differences of the velocity of the torso.

particularly, in detecting of a beginning of a fall which is a mixed fall of a tilt fall and a vertical fall, a combined mixed fall beginning threshold value can be used which comprises a portion that depends on a vertical fall velocity and comprises a portion that depends on a threshold value for the beginning of a tilt fall which defines a threshold for movement and/or position of a projection point in the standing basis. The projection point is described in detail below. Analoguously, a mixed ground fall threshold value can be defined.

Further, it is possible to recognize a beginning of a fall and a ground fall from a velocity in direction of a ground plane which is defined in detail below or from an expected impact seed. This is possible for all types of falls. The current velocity of torso, head or wrists, for example, can be compared with a threshold value for velocity and direction of the ground plane, respectively.

In this patent application, the center of gravity is a fictitious point in which the mass of the body of the human is considered as concentrated. The center of gravity differs from the actual center of mass in that the position of the center of gravity and possibly also the mass is an approximation to reality. As the actual center of mass this difficult to determine and can move by variations of the posture of the body, the center of gravity can be used as an approximation instead.

The standing basis is a fictitious area in which an active support of the center of gravity can take place by force transfer into the ground on which the human is located. Additionally, the standing basis can also comprise areas in which no force transfer into the ground takes place, but which can be relevant for the calculation of the support of the center of gravity in the past or in the future. The thoughts regarding the standing basis which are described in the following can also be applied to a standing basis through the ankles as far as the form of a foot or a shoe is not relevant. “Foot” or “shoe” is then to be replaced by “ankle”. The standing basis can comprise an area under a foot which is set on the ground and/or comprise an area under a foot which is not set on the ground, as there is a probability that it will be set on the ground in order to take over the support of the center of gravity, for example when walking or when cushioning a fall. Furthermore, the standing basis can comprise an intermediate area between two feet which are set on the ground. A standing basis can have a two-dimensional extension which is, however, not required compellingly. A standing basis can be a connected area; however, it can also comprise non-connected areas and/or sections. The standing basis can comprise a standing basis area and/or a standing basis line and/or a standing basis point which define the standing basis. A standing basis line can for example represent an approximation of the reality or map that a human, for instance, wears ski, skates or in-line skates.

The standing basis is located in a fictitious ground plane that is defined to this purpose and which can coincide with a real ground plane or can approximate it. The ground plane does not have to be a connected area; however, this possible. All places on which the human can support body weight by a foot, or approximations thereto, can be valid as a ground plane. It is also possible that a human supports weight at places which do not belong to the standing basis, for example, in case that he holds on somewhere or supports himself at another place of his body except of the feet.

In order to detect a fall risk or a fall, in a simplifying manner, a standing basis can be used which is not the actual support area of the feet but extends through the ankles which, in many situations, have a constant relative position to the actual support area. Said calculation of connection information on basis of the ankles can be part of a more extensive calculation which also comprises more parts of the calculation. For example, the calculation can further take into account the relative position between the ankles and the undersides of the feet or the soles of the shoes such that a standing basis can be used which represents the actual support area.

A standing basis is a fictitious value and can also comprise a section in which the human does actually not support himself. This can be the case because of approximations of a real support area by the standing basis area. The standing basis area also does not have to comprise the whole real support area, for the same reason. Also, the whole standing basis area can be a purely fictitious standing basis area on which the human does not stand at all, for example, when he has jumped off, runs in which, in phases, both feet are lifted from the ground or loses ground contact in a fall.

A standing basis area can be defined in a ground plane even if the feed do not touch the ground, for example from data of the humans course of lifting off from the ground. When both feet are lifted off the ground, the ground plane is preferably located in a distance to the human in which he should theoretically be located relative to the ground area which can be a result of a jump height, a jump trajectory, a fall height, for a time period since the loss of ground contact to a ground plane and/or velocity and/or an acceleration as well as their directions during loss of ground contact and/or a flight path The ground plane can approximate a real ground plane, particularly regarding height and/or a tilt angle. It can also coincide with a real ground plane or partially coincide with it. At least one foot or a shoe which is connected with it can be set on the ground plane in the presence and/or can have been set to it in the past and/or can, presumably, be set to it in the future.

A projection of the first or second foot or of the footwear of the first or the second foot or an underside of it into the ground plane as a part of the standing basis area can be carried out in direction of gravity, or in direction of movement of the foot, or in direction of the acceleration of the center of gravity, or along a path of free flight without assumption of influence of forces, or along a swiveling line which results of swiveling the body around the ankle of a foot which is set on ground, or along a typical movement which is actively carry out. The proposed possibilities can be combined with each other. Inter alia, they can be applied succeeding each other in time. For example, in situations in which a human walks or runs, swiveling around the foot which is set on ground can be assumed which can be used as a basis for the projection line. When jumping of falling, for example the path of free flight can be used as the projection line along which no forces except gravity act onto the body. A projection in direction of gravity is less calculation power demanding and simulates the situation in which the foot would fall down without consideration of further forces or velocities. A projection in direction of gravity demands less calculation efforts and simulates the situation in which the foot would fall down without consideration of further forces or velocities. A projection in the direction of movement of the foot is preferably applied in a time period before setting it to the ground. Further, it is conceivable to use as projection line a typical line of movement of a foot when walking or running which can, for example, be adapted to the walking or running seed.

The standing basis area particularly comprises:
if a first foot or a footwear of the first foot is set on a ground plane, an underside of a first foot or a footwear of the first foot as the first foot standing basis area, and
if the first foot or footwear of the first foot is lifted off from the ground plane, a projection of the underside of the first foot or a footwear of the first foot into the ground plane as a first foot standing basis area, and
if a second foot or footwear of the second foot is set on a ground plane, an underside of the second foot or a footwear of the second foot as the second foot standing basis area, and
if a second foot or footwear of the second foot is lifted off from the ground plane, a projection of the underside of the second foot or a footwear of the first foot into the ground plane as a second foot standing basis area, and
an intermediate area of the standing basis between the first foot standing basis area and the second foot standing basis area.

A standing basis area which is formed by a foot can be approximated, as the accurate determination of the standing basis area can be difficult and it is not worth the effort in many cases. Preferably, the location of the border section of the standing basis area for comparison with the projection point is defined as at least one section of an outline contour of the feet or of a connected shoe which is not a border section at which the feet are facing each other and/or a location of the standing basis area defined by a connecting line which touches the outline contour of the both feet at least approximately tangentially. The connecting line is preferably straight.

When a foot is partially set on ground, for example when stepping on a step of stairs or on a curb or when rolling the foot, the standing basis area can be reduced to an area which comprises only the part under the foot which is set on ground. It is possible to detect at least a part of a border of this setting-on-ground area under the foot, at least approximately, for example by detecting a few, for example of four, sectors under the sole of the foot. However, preferably, for a more safe and accurate detection of the risk of fall, the situation and position of the standing basis area is determined with a good accuracy, for example less than 80 mm, preferably less than 40 mm and most referred less than 20 mm. The invention can also be carried out with an accuracy between these two extreme values. For detecting the setting-on-ground area, a sole of the shoe, an insole or a sock which is equipped with pressure sensors can for example be used which is able to detect which part of the soul is part of the setting-non-ground area. Regarding the rolling of the foot, a part of the foot which is set on ground can be estimated from the posture of the leg. After determination of the process of walking, also a part of the foot which is set on the ground can be estimated from the phase of the walking process.

The standing basis area can at least approximately be oriented perpendicular to the direction of gravity, for example, if the human stands on a horizontal and even, real ground plane. However, the standing basis area can also be oriented inclined to gravity if somebody stands at a sloe or in a terrain with uneven ground or walks along there. By carrying out a projection in direction of gravity during the proposed detection of a risk of fall or a fall, an increased tendency of tilting over at a sloe or an uneven ground section is represented in that the standing basis area appears to be shortened by its inclination.

However, for a simplification of the method, taking into account the inclination of the actual standing basis area can be relinquished.

An inclination angle of a foot or a shoe can for example be measured in relation to gravity by an inclination sensor or multi axis acceleration sensor. It is also conceivable to locate an angle sensor at the ankle which detects an angle between the lower leg and the foot, wherein, preferably, additionally the angular position of the lower leg in relation to gravity is known or measured. However, it can also be resumed that the angle of the lower leg, at least when walking and at least approximately, is located in a plane which is defined by the longitudinal direction which means in direction of front and back and the direction of gravity. Then, an inclination to the side of a real ground area can be detected only from the measured angle between the lower leg and foot. If the inclination angle of the foot or shoe which is set on the ground is known in relation to gravity, the angular position of the associated standing basis area can be calculated as parallel to a foot sole or a shoe sole of the foot. A projection of the real foot soles into the standing basis area can be calculated along direction of gravity or along a perpendicular direction to the standing basis area. Preferably, the ground area is calculated such that the angular deviation relative to the angles of the foot soles is minimal in sum. The standing basis area can, at the place at which it is located, have the form of the ground area at this location. If the foot soles are not oriented in parallel to each other which is, inter alia, possible by tilting the feet, the ground area is preferably calculated with consideration of the tilt of the feet, for example as a curved area or as sections of even planes.

However, it is also possible that the angular position of the foot sole is unknown. particularly, if two feet or shoes are set on the ground, the ground area can, in regard of its angular orientation, be defined as a connecting plane of foot points of the feet or shoes or sorts footwear or previous positions of the aforementioned. A foot point is a point, preferably a midpoint, at the underside of the foot for the shoe or the sorts footwear such as a roller skate, a skate, a ski, a snow shoe or the like. A setting-on-ground point is a foot point of a foot which is set on ground. Also, a foot point can be used which is not a current setting-on-ground point and, particularly, has been a setting-on-ground point in the past. Preferably, two foot points are used in the ground plane in order to define a first direction of the ground plane. A second direction in a ground plane which is defined in such a way, can be a perpendicular direction to the direction of gravity in order to define an orientation in space, particularly if further information regarding its inclination is lacking. Alternatively or additionally, it is conceivable to define a further direction of the ground plane by a current and last setting-on-ground point of the same or another foot. Also, a ground plane can be calculated as a plane from three foot points. Three points define a mathematical plane space. Preferably, at least one point which is used for defining the plane is a current setting-on-ground point of a foot. If both feet are set on the ground, their set-on-ground points and the last set-on-ground point of one of the feet can be used for calculation of the ground plane. Further, it is conceivable to define the ground plane by one current and the two last setting-on-ground points, or, in case without ground contact at both feet, from the last three setting-on-ground point. An anlogon can be applied when defining the ground plane by directions.

It is further conceivable that the ground plane is not a plane in a mathematical sense, but can also contain curved sections or steps. This provides a higher accuracy in the recognition of a risk for and/or its consequences. Such a ground plane can, for example, the captured by one or more cameras, especially as a three-dimensional surface. At least one setting-on-ground point of at least one foot and/or measurement points from a contactless measurement with, for example, an ultrasonic sensor and/or a photonically operating time-of-flight measurement system can also be taken into account. It is also conceivable to define such a complex ground plane from several partial ground planes which can be defined by single foot points and/or measurement points and possibly by their environment.

If, for example, a foot or a shoe is off the ground when walking or at a beginning fall, a ground plane can be calculated for using it in monitoring of the walking or of the use of sorts devices which are connected to the feet. The contour of the lifted off foot or footwear or a setting-on-ground area of the sorts device or an approximation thereto can be projected into the ground plane in direction of gravity or in direction of the velocity of the foot or along an expected movement of the foot, in order to form an expected standing basis area there which is realized when the foot is set on the ground. From the regularity of the arrangement of the standing basis area of the feet, possibly also from the past, can be estimated if a normal walking processes is present or if the fall can still be prevented by a catching reaction, respectively. A further possibility of this assessment lies in the verification whether the projected point of gravity lies in the expected standing basis area in the next step.

The human body can, in a theoretical approximation, be considered as an upside-down, upward directed pendulum, the center of gravity of which is pivoted unstably above a pivoting point. If such a pendulum starts tilting, it initially tilts very slowly and then accelerates increasingly more until the impact. For this reason, at the beginning of a fall of a human, time is left in order to prevent the impact by a catching reaction. Typically, this is a reflex in which at least one foot is brought in a direction in which the body falls in order to stop the falling over. During this, if the catching reaction is successful, the standing basis is brought in orientation with the center of gravity along the current acceleration of the center of gravity towards the standing basis again. Particularly, the standing basis is extended by a lunge step in such a way that it supports the center of gravity again. If the projected center of gravity is in a distance to the expected standing basis area, a beginning of a fall is present, wherein a ground fall, wherein falling to the ground is expected, is or appears to be avoidable if the projected center of gravity is supportable by the standing basis area with a catching reflex such as a lunge step or a step which differs in another way, such that the person does not hit. For example, an expectation for a viable deviation from the expected standing basis area on a catching reflex can be defined, particularly depending on the bodily condition of the person.

Fundamentally, the method is based on the principle that the human is in balance and does not fall by tilting if, starting at the center of gravity of his body, in direction of an acceleration of center of gravity a standing basis is located. For detecting whether a tilt fall or a risk of a tilt fall is present, it can be determined to which grade this is correct. A risk of fall is present if the support of the center of mass by the standing basis in direction of acceleration of the center of gravity is threatening to get lost. A beginning or advanced fall is present if this support is lost. An amount of the advancement of the fall can be detected by how far the relative position of the center of gravity and the standing basis has moved away from the direction of the acceleration of the center of gravity which can be determined in different ways.

Specifically, the tilting of a human can start if, in direction of the acceleration of the center of gravity, the center of gravity which is projected into the standing basis moves over the border of a foot or a shoe. If the human wears as a footwear a shoe or a sorts device which is indicated as a footwear in this patent application as well, such as a ski, an in-line skate shoe or a skate, or rides on a snowboard or a skateboard, the setting-on-ground area of the foot can be increased or reduced and extend to the border of the foot wear. For example, if a human stands with legs apart and tilts over to the front, however, the center of gravity does, in consequence, not move over the foot but over an intermediate area between the both feet which normally belongs to this standing basis area as well. This intermediate area, in this case, is spanned between between the tips of the feet and the ends of the heels and ends at the inner sides of the feet or the shoes, respectively. The limitation of the standing basis area to the front and to the back, in this case, is a connecting line between the big toes and the ends of the heels. The intermediate area can be defined in a similar way for further conceivable relative positions of the feet in relation to each other. It can be taken into account that a foot can also be turned. Then, the connecting lines can, under certain circumstances, begin and end at other places and also at places at the feet that differ from each other. As an outline contour of the intermediate area, a straight connecting line is preferably placed against the outline contour such that it connects the contours of both feet, wherein prolongation of the connecting line does not run through the part of the standing basis area, which is located under the feet. Then, the connecting line is placed against the outline contour of both feet at least approximately tangentially. This is true for both connecting lines. The expression “at least approximately tangentially” comprises that the connecting line can be exactly tangential as well as the case in which the connecting lines are placed against a protruding radius in the contour of a foot sole. The meaning of the term “tangential” shall, in an analoguous sense, also be valid for other features which are described at other passages in this patent application as “tangential”.

The presence, particularly also the grade of a risk of fall, and/or the presence of a fall, particularly of a beginning or an advanced fall, can be detected by calculation inside the standing basis or the ground plane into which the center of gravity is projected by calculation, or by calculation inside a plane which extends through the center of gravity of the human and into which at least one point of the border of the standing basis area is projected against the direction of the acceleration of the center of gravity. Further, the ground plane and/or others standing basis can be used in case of a fall in order to calculate the point in time and/or a vehemence of impact in advance. For this purpose, a distance of the center of gravity from the ground plane can be calculated and used and/or a current or a velocity that has been calculated for the impact in advance can be used. If, for example, the ground plane is heavily inclined, the impact can be milder if the human falls into an ascending sloe, but can be vehement if the human falls down the sloe. The same is analoguously true for stairs. Stairs can be approximated by a ground plane, wherein it is possible to neglect the steps and for example define them as an inclined plane. The plane can extend through a foot point of a foot which is set on the real ground. A plane onto which an impact is to be expected and which is higher or lower as the height from which the fall begins, a can be taken into account in a calculation of the vehemence of the impact, if it is known, for example, because the person has stood on it before or because it has been detected by a measurement, for example by ultrasonic distance measurement technology, laser distance measurement technology or video technology, particularly 3D video technology. The vehemence of the impact can be taken into account in a decision regarding a triggering of measures for protection. For example, the triggering of measures for protection can be dispensed of if a low vehemence of impact is expected, for example, below are threshold value which can be adapted depending on the susceptibility for injuries of the falling person. For example, such an adaptation for a young sportsman can differ from an adaptation for a senior lady.

The influence of an acceleration of the center of gravity at the center of gravity means, in this patent application, a fictitious process. Actually, accelerations are effective at many places of the body, the total effect of which is thought as influencing the center of gravity. Therefore, it can be difficult to determine the acceleration at the center of gravity. Thus, an approximation thereto can be used.

The acceleration of the center of gravity preferably comprises the gravity acceleration at the center of gravity. Preferably, the acceleration of the center of gravity comprises additionally an acceleration of the center of gravity in space and/or a centrifugal acceleration at the center of gravity. If a human changes his velocity, also an acceleration of the center of gravity in space is effective besides of the gravity. If a human underlies a rotation with a center of rotation which is outside of the center of gravity, the centrifugal acceleration can be effective on his center of gravity. If he is located in a rotating system, additionally Coriolis accelerations can be effective on his center of gravity.

Taking into account an acceleration of the center of gravity in space and/or a centrifugal acceleration can particularly be carried out if the human underlies corresponding accelerations. In activities as sitting, standing or lying, it can be relinquished. However, it is possible though, to use only gravity acceleration has a simplification. Breaking forces are considered as negative accelerating forces, as usual.

It is proposed to project a center of gravity of the human in direction of the acceleration of the center of gravity into a standing basis area of the human as projection plane or to project the standing basis area or at least a part of the border of the standing basis area into a projection plane in which the center of gravity lies, and which is arranged in an angle to the acceleration of the center of gravity, which preferably is 90°. The projection of the center of gravity in direction of the acceleration of the center of gravity into the standing basis area will be called a projection point in the following. The same principle can be realized by not projecting the center of gravity of the human into the standing basis area, but, the other way round, the standing basis area or a part of it is projected into a plane in which the center of gravity is located. The projections each follow the direction of the acceleration of the center of gravity or its adverse direction, respectively, and are to be considered as connections for determining balance. In the following, for reasons of simplicity, it can be spoken of a projection of the center of gravity into the standing basis area; however, it is always meant also the analogue solution mentioned above, according to which the standing basis area or a part of it is projected into a plane comprising the center of gravity.

Preferably, a reference location of a border of the standing basis area is determined in the projection plane. A risk of a tilt fall or a tilt fall can be derived from a position of the projection point in relation to the border of the standing basis area and, particularly, in relation to the reference location.

A reference location can be a section of the border of the standing basis area. A reference location is chosen preferably for recognizing risk of fall or fall, in order to determine how close a human is to a limit of his balance and/or whether this is already exceeded. The position of the reference location on the border of the standing basis area is preferably associated to the direction in which the human is threatened to tilt or tilts. The reference location can for example be chosen as a location of the border of the standing basis area at which a connection line between the reference location and the border of the standing basis area forms a normal line to the direction of the course of the border of the standing basis area. This can be ambiguous and deliver several reference locations. Preferably, the reference point is chosen which is the closest to the projection point or to the center of gravity, and most preferably, a reference point is chosen which is located at least approximately in direction of the movement of the center of gravity.

Preferably, the projection is carried out by calculation. From at least two subsequent of such calculations, the velocity of the projection point or the center of gravity can be determined in relation to the standing basis area or its border or the reference location, and from three of such calculations, the acceleration of the projection point.

Particularly, a risk of a tilt fall is detected if the projection point in the projection plane is located inside of the standing basis area and the projection point is located closer as a fall risk distance at the border of the standing basis area and, particularly, at the reference location, and/or the projection point moves with a relative velocity towards the border of the standing basis area or towards the reference location on the border of the standing basis area or towards the reference location that makes expect that the border of the standing basis area or the reference location is reached in less than a redefined time, respectively, and/or the relative velocity is above a fall risk threshold value.

A tilt fall can be detected, if, in the projection plane, the projection point is located outside the standing basis. For this, the beginning of a tilt fall is preferably detected if the distance of the protection point from the border of the standing basis area or from the reference location is less than a ground fall threshold distance and/or the relative velocity of the protection point is less than a ground fall relative velocity and/or a relative acceleration is less than a ground fall relative acceleration. Alternatively or additionally, a ground fall can be recognized if the distance of the projection point to the border of the standing basis area or to the reference location is greater than the ground fall threshold distance and/or the relative velocity of the protection point is greater than the ground fall relative velocity and/or a relative acceleration is greater than a ground fall relative acceleration.

If the projection point is located in the standing basis area or, analogously thereto, the center of gravity is located inside as standing basis area which is projected into a projection plane, and the human is in balance, torque around the transverse axis of the human is required in order to cause him to fall. Such a torque can for example arise if the human receives a shove or runs against an obstacle. By this, the center of gravity is moved in relation to the standing basis area. If the projection point moves out of the standing basis area, the human gets into an unstable state. The torque which acts on the human effects that the human starts tilting which results in a further increase of the torque such that the tilting process gains velocity progressively. A leaving of the stable position can also take place if the standing basis area under the center of gravity varies such that the projection point is not in the standing basis area any more. The standing basis area can vary, if the feet are varied in their position in relation to the projection point. When walking, the standing basis area varies with each step. Walking is a dynamic process in which temporarily only one foot is on the ground. In this situation, the human is threatened to tilt over with each step, in principle; however, walking is such a type of dynamic process that the process of tilting over is caught each time by setting the other foot on the ground. For this, the foot has to be set on the ground in the right position. One can also span the conceived standing basis area between a projection into the ground plane of a foot that is lifted off the ground and the foot that is set on the ground and use it for fall recognition. In a working dynamic walking process, the projection point remains in a standing basis area that is spanned in this way. However, it leaves the standing basis area when braking and when accelerating as a leaning back or leaning forward is indispensable for those variations of velocity for reasons of balance. However, this is re-caught by corresponding steps, normally.

If the projection point is located inside the standing basis area, but at the border of the standing basis area or at the reference location, a risk of fall this present. If the projection point is located outside of the standing basis area, then the human is at least at the beginning of a fall. In general, the fall is advanced the further, the farther the projection point moves away from the standing basis area. particularly, if the projection point moves from inside the standing basis area towards towards the border of the standing basis area or the reference location, it is to be feared that this movement is continued and the human gets into the region of a fall. The movement of the projection point in the standing basis area represents the tilting of the human in a certain direction which is associated with a mass moment of inertia such that a continuation takes place without further application of force. The movement of the projection point is therefore especially relevant for the point in time at which the human becomes endangered for a fall in this direction and/or whether a fall in this direction occurs. In this, it is particularly interesting whether a beginning of a fall at which the fall can still be caught, or a ground fall takes place at which a catching is not to be expected any more. For this, the location of crossing the border of the basis over which the protection point or center of gravity, respectively, will expectedly move, is preferably determined. Alternatively, it is also possible to detect the complete border of the standing basis area. Preferably, the recognition or detection, respectively, of the border of the standing basis area is repeated many times in order to monitor the current situation of the human. The repetition rate of recognition or detection, respectively, can be adapted to the situation of the human and can, for example, be carried out more often when walking, jumping, standing up, sitting down, ascending stairs or a step or at recognition of a risk of fall or a fall as in other situations. The repetition rate can be carried out depending on the grade of risk of fall, particularly depending on the distance of the projection point or the center of gravity, respectively, from the border of the standing basis area or the location of crossing or a reference location. A location of crossing or reference location of the border of the standing basis area can be a point or a section of an outline contour of a foot or of a shoe which is connected to it or of an underside thereof or the border of the intermediate area.

When stumbling at which, when walking, one foot gets stuck at an obstacle, the human cannot bring, under certain circumstances, the standing basis area under the center of gravity any more in order to re-catch the controlled fall in putting up one foot. In this, if balance gets lost, the projection point moves from inside of the standing basis area over the border of the standing basis area in the surroundings of the standing basis area. Normally, the foot which gets stuck is the foot which is moved over the ground plane and which is thus not loaded with the weight of the body. A catching reflex has therefore quite good chances to succeed, particularly, if being stuck can be ended. Whether a fall actually takes place, can be assessed early by the method of movement of the projection point or the center of gravity, respectively, out of the standing basis area as described here. Particularly, continuing being stuck can be detected. This can be detected by a fixation of the standing basis area of the stuck foot, particularly, if the person is in an imbalanced state, additionally.

When slipping, the friction between the location of setting the foot on the ground and the foot gets lost, such that it can slide away to the side without control. In this, the foot normally supports a significant part of the weight of the human. Like in stumbling, the standing basis area is varied uncontrolled which can make it impossible to bring back the projection point to the standing basis area. When slipping, this is exacerbated in that at least one leg has to be replaced which is supporting at least partially, wherein the place at which the foot that is slipping away has been standing originally, is slippery. Whether a fall actually will take place, can be assessed early with the method of the movement of the projection point out of the standing basis area that is described here.

The absence of a required catching step can be recognized if the standing basis is fixed at least partially and the distance of the projection point from the border of the basis area increases.

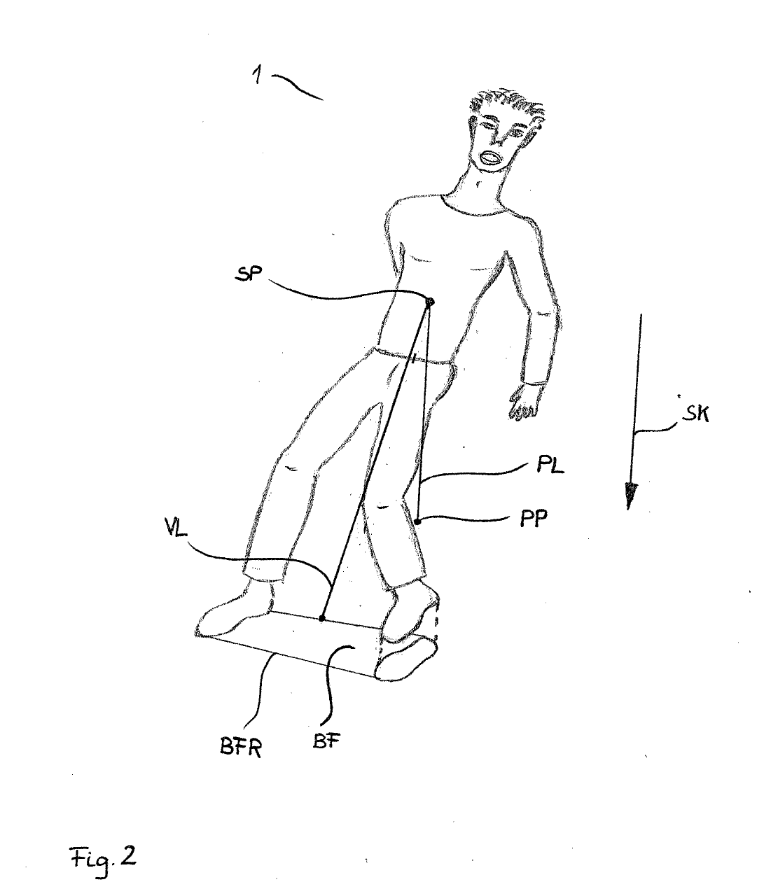

Further, a method is proposed in which the difference of directions between the reference line from the center of gravity to a reference location at the border of the standing basis area and the direction of an acceleration of the center of gravity is detected. From the difference of directions, a risk of the tilt fall or a tilt fall can be derived. The recognition of a risk of fall, the beginning of a fall and a ground fall which is described in this patent application in relation to a projection point can be adopted analoguously. It is the same detection by another geometry. However, the same principle is applied. A drawing of the principle is shown in FIG. 2.

Preferably, at least one airbag is opened if a ground fall is recognized. This can be carried out in all methods for recognizing a ground fall. An airbag is compacted in a not activated state and can then be opened by activation, wherein it enlarges significantly and is capable to cushion an impact. For example, the airbag can be inflated or be opened by a spring mechanism.

It is proposed that the connection information regarding the connection between the standing basis and the center of gravity is detected from a variable portion of variation and optionally additionally from a non-variable fixed portion, wherein, particularly, the variable portion of the connection between the standing basis and the center of gravity located between a foot approximation location close to the foot, particularly at the ankle or at the foot, and a center of gravity approximation location at the torso of the human and, particularly, close to the center of gravity of the human influences the detection of the connection information, and particularly, the non-variable fixed portion of the connection between the standing basis and the center of gravity located between the foot approximation location and an associated foot standing basis area and between the center of gravity approximation location and the center of gravity influences the detection of the connection information.

The fixed portion of the connection between the standing basis area and the center of gravity comprises the section between the standing basis area and the foot approximation location. The underside of a foot or a footwear can be detected by a linear movement of their coordinate depending on the position of the foot approximation locations, wherein a fixed distance between the foot approximation location and the underside can be included into calculation. Preferably, also the orientation of the underside of the foot is constant. As in most cases the feet are set on the ground with the underside perpendicular to the gravity, this is a suitable solution with few calculation effort for many cases. However, if information regarding tilt of the foot is present, it can be taken into account by a rotational transformation of the coordinates of the underside. particularly, rotations around the longitudinal axis of the lower leg can be taken into account. Such rotations have effects on the location of the standing basis area which can be optimized in sense of a more accurate detection of the risk of fall of a fall in this way. In sense of simplification and reduction of the calculation efforts it is also conceivable not to calculate the standing basis area explicitly, but to assume it is a standing basis in form of a standing basis line between the foot approximation locations. In this way, a rough recognition of the balance or the risk of fall or a fall of a human can be carried out, respectively, wherein the risk of fall is then not recognized by the criterion that a projection point in the standing basis area or the direction of the acceleration of the center of gravity from the center of gravity in comparison to the direction of the connecting line is directed further towards the standing basis area, but by the grade of distance of the projection point or by the grade of the direction deviation from the connecting line. The above-mentioned features of a standing basis area in regard of a lifted foot and a projection into the ground plane can be realized analoguously. A foot approximation location is preferably arranged at or close to a lower end of a lower leg or in the ankle, respectively. An advantage thereof is that the position of this location at the lower leg is close to the foot and the position of the lower leg in relation to the center of gravity approximation location can be calculated relatively easy. In the detection of the standing basis, the size of a foot or a footwear can be taken into account. Besides the footwear socks and shoes, also roller skates, ski, snow shoes and skates, snowboards and skateboards are considered as functional footwear, for example.

The fixed portion of the connection between the standing basis area and the center of gravity comprises the section between the center of gravity and the center of gravity approximation location. This section can be defined as a fixed distance in a redefined direction from the center of gravity approximation location. The redefined direction can be dependent on a direction which is present at the center of gravity approximation location towards the center of gravity, wherein the redefined direction can at least roughly extend in longitudinal direction of the torso. Preferably, the center of gravity approximation location is located at the lower end of the torso, but above the hip joint. Preferably, the center of gravity approximation location is located above the hip. This has the advantage that the location of this position in regard of the standing basis is detected by the method for detecting the variable portion which is explained below. However, alternatively, also other methods and other center of gravity approximation locations possible. However, other methods and other center of gravity approximation locations are possible, alternatively. It is conceivable to use several different center of gravity approximation locations which, particularly, can be associated to one foot which is set on the ground, respectively. The accuracy of a detected position at the hip can depend on the foot from which it is calculated. Thus, by the change of the center of gravity approximation locations, accuracy can be increased. A center of gravity approximation location can approximate the center of gravity or be identical with it. Then, the above-mentioned fixed distance between the centers of gravity of a center of gravity approximation location is zero. The center of gravity can approximate the actual center of mass of the body of the human or be identical with it. For example, the center of gravity can be located inside the body close to the navel. The direction and the distance of a fixed portion to the center of gravity can, for better accuracy, depend of the posture of the body in order to take into account movements of the center of mass, for example when bending down.

The variable portion can be detected by using data from one or more sensors which directly or indirectly measure the bending of the knee, and by using data from one or more sensors which measure directly or indirectly the posture of a hip joint, and by using lengths of the lower legs and the thighs. In this way, connecting information between the torso and the lower leg of the human can be obtained.

Particularly, for an indirect measurement, an inclination sensor for measuring the inclination in regard of the gravity and/or an acceleration sensor with static measurement function can be used, which are, preferably, integrated into garment and/or, preferably, interference accelerations from a rotation of a part of the body with which the inclination sensors or acceleration sensors are connected mechanically for measurement, are compensated for by using an angular rate sensor and/or, for direct measurement, angle sensors are used which are, for measuring an angle of parts of the body in regard to each other, connected to these parts of the body and are, particularly, integrated into garment or into a harness for wearing by a human.

preferably, a multi-axis inclination sensor is used which is capable to measure the orientation of the sensor in regard of gravity in two measurements directions. Alternatively or additionally, a multi-axis acceleration sensor is used, which is able to measure acceleration in two, or particularly referred in three directions which are orthogonal to each other.

The human body comprises almost exclusively joints which act as a pivoting joints. Thus, it is possible to determine the varying portion of the relative position, wherein the varying portion varies by movements of the human, by determining the angular positions of the involved parts of the body. Taking into account the lengths of thighs and lower legs, connection information can be calculated from the measured angles which concern the relative position of the hip and the ankle. For this, an angle of the thigh in regard of the lower leg, thus a knee angle, and an angle between the thigh and the torso, thus a hip angle, can be measured. As a model for the possibility of movements of the human, a joint with one rotational degree of freedom at the knee and joint with three rotational degrees of freedom at the hip can be assumed. Alternatively, for simplification, it is also possible to assume only two degrees of freedom at the hip, wherein the rotational degree of freedom around the longitudinal axis of the leg can be omitted. Furthermore, for the purpose of a more accurate detection of a risk of a tilt fall or a tilt fall, the torso can be divided into two sections which are arranged over each other during standing upright which are tiltable in regard to each other, corresponding, for example, to a rolling the torso to the front.

In order to recognize a risk of a tilt fall, it is required to determine the standing basis, the connection information between the center of gravity and the standing basis as well as the direction of the accelerations at the center of gravity. These values are variable and depend on the current posture of the human. For detecting the standing basis, the relative position of the feet to each other can be detected by measurement and, if required, by post-processing of measurement data by calculation. The connection information between the center of gravity and the standing basis can be detected in an analoguous way. Finally, the direction of the acceleration at the center of gravity at the center of gravity or close to it can be measured by an acceleration sensor, or it can be concluded from other data to them by calculation. particularly, these data are present from the detection of the standing basis and/or from the detection of the connection information regarding the spatial connection between standing basis area and center of gravity.

An angle of the thigh to the lower leg, thus a knee angle, and an angle between a thigh and the torso, thus a hip angle, can be measured indirectly by relating the angle positions of the thigh, the lower leg or the torso to the direction of gravity and by associating those angles to each other, and, particularly, to subtract them from each other. Preferably, angle and/or acceleration sensors are used which are measuring multi-dimensionally. By an acceleration sensor, as a result of a static measurement, its inclination in regard of the acceleration of gravity can be detected. However, this is only possible if the time intervals in which a newly measured inclination shall be available are not similar to the time periods of typical movements which, however, presently is the case most times. A compensation of effects of movement is thus desirable.

If inclination sensors or acceleration sensors are used in order to measure an angle of a thigh, a lower leg or a torso of the human in relation to the direction of gravity, the sensors are not only exposed to gravity, but also measure, in superimposition, further accelerations on the part of the body, such as accelerations of the part of the body, centrifugal forces. These forces interfere in the determination of the angle in relation to gravity. In order to come to the angle in relation to gravity, though, these disturbance variables can be compensated for.

If an acceleration sensor is used for compensation, it is helpful for such compensation to have a fixed reference point at which only gravity is effective. This is the case at a foot which is set on the ground. Preferably, an acceleration sensor is fastened to the foot or the ankle or at the lower end of the lower leg with which can be detected whether the foot is set on the ground. This is usually the case if only accelerations are present which are small or which are typical for a foot which is set on the ground in a walking movement. corresponding detection methods are also known from gait recognition.

Preferably, for detecting a foot which is set on the ground, a sock, a pantyhose, an insole, a shoe sole, an anklet belt for the ankle and/or a hem of a pair of trousers is used which preferably comprise an acceleration sensor, a pressure sensor and/or an inclination sensor. When using a shoe, a sock, a sole, an independent anklet belt, a radio connection to a calculation device which can be arranged in a pair of trousers or in a garment for the torso can be present. A cuff with a sensor can be provided which is fastened to a hem at a leg of a pair of trousers and which elastically touches the ankle or the lower part of the lower leg. However, it is also possible to use a loose hem and to fix a sensor there. The hem is located close to the foot or the ankle. When fixing at a garment for the legs, a radio connection can be dispensed with in many cases. Whether a foot is set on the ground can be detected, for example, in that accelerations are present which are only small or which are typical for a foot that is set on the ground in a walking movement. corresponding recognition methods are known from gait recognition.

Starting from the foot that is set on the ground, a position and/or velocity of the knee can be calculated via an angular position and/or an angular velocity and/or an angular acceleration of the lower leg in connection with the length of the lower leg. The angular position can contribute to the calculation of the standing basis area as well as to the determination of the variable portion of the relative position between this standing basis area and the center of gravity. If an acceleration sensor or an inclination sensor is used which is, for example, arranged in the middle of the lower leg, its measurement of the angular acceleration or other angular velocity, respectively, can be disturbed in that it underlies an interfering acceleration by a movement of the lower leg. A self-compensation is possible, but leads to high calculation effort because of the requirement to solve a non-linear equation. Also, a compensation with an angular rate sensor and a magnetic field sensor is possible. The content of the US 2007/0032748 regarding this shall be included into this patent application.

In order to work around this problem, it is proposed to arrange an acceleration sensor or an inclination sensor a few above the ankle where practically no accelerations except gravity are present if the foot is set on the ground. However, the variation of the angle of the lower leg can be measured here. Then, the position and the velocity of the knee can be calculated without compensation of accelerations or centrifugal forces at the sensor at the lower leg and exclusively via the geometry of the lower leg. The angular velocity of the lower leg can be detected using an acceleration sensor, however, it is preferably detected by an angular rate sensor at the lower leg.

The same principle can be applied in similar form once again if an acceleration sensor or an inclination sensor is arranged above and close to the knee, for example in less than a distance of 12 cm from a fictitious pivoting point of the knee, in order to detect the angle of the thigh. Then, only small accelerations by the rotation of the thigh are present at this sensor, such that a compensation for movements of the thigh can be relinquished. A compensation of the accelerations at the knee is sufficient which is known by the movement of the lower thigh. Here, a centrifugal acceleration from the rotation of the lower leg and angular accelerations of the lower leg are effective. By the compensation, the acceleration of gravity can be detected without interference of disturbing accelerations and be used for detecting the angle of the five. Because of the known position of the knee, the angular position which is measured above the knee and the movement of the thigh, the movement of the hip joint can be calculated in a simple way.

The same principle can be used multiple times in form of a chain by arranging an acceleration sensor or an inclination sensor directly above the hip joint of which the position has been detected with the last mentioned acceleration or inclination sensor, a further one directly below the other hip joint at the thigh and yet a further one directly below the other knee. In this way, the position of the other ankle from which the calculation has not begun, can be calculated. As then both positions of the feet relative to each other are known, the position of the standing basis can be determined.

When walking, the standing foot is alternating such that it is proposed to establish the chain of acceleration and/or inclination sensors that is free of self-compensation also beginning from the other foot or ankle, respectively. Then, in total 10 acceleration and/or inclination sensors at both legs and his result, wherein one is arranged above and below of each joint, respectively, and one above and below of each hip joint, respectively, as well as above of each ankle.

It is also possible to carry out a compensation by an angular rate sensor which is connected to the same part of the body with which also the acceleration sensor is connected which is to be compensated. Measurement results of the angular velocity and angular accelerations which are derived thereof can be used in order to compensate a compensation of interfering accelerations from the rotation. This concerns accelerations from centrifugal forces and angular accelerations. For the compensation, the distance of the location for which compensation shall be carried out from the pivoting point must be known. When compensating interfering accelerations at parts of the human body, rotations take place in the joints as pivoting points. The distance of acceleration sensors at which interfering accelerations shall be compensated for to the joints is normally known from the position in the fall recognition device which may have the form of the garment. For compensation, a centrifugal acceleration can be calculated as a product of the distance of the acceleration sensor from the joint and the square of the angular velocity and can be subtracted vectorially from the acceleration which has been measured with the acceleration sensor. An angular acceleration can be compensated for by a product of the derivation of the angular velocity multiplied by the distance between the acceleration sensor and the joint. An advantage is that the compensation can take place with only few calculation effort, independently of the position of the acceleration sensor at the part of the body.

Even with compensation by an angular rate sensor, the acceleration sensor can, additionally, be subject of a translational acceleration of the whole part of the body. This can, for detecting the direction of gravity relative to the acceleration sensor, be compensated for, too. This is, as described before, possible in that the direction of gravity and the movement are detected first at a lower leg of which the foot is set onto the ground. In difference to the compensation method described above it is, however, it is not required here to arrange the angular rate sensor and/or the acceleration sensor at the ankle, but it can also be arranged at another place at the lower leg, particularly not far below the knee. The rotation of the lower leg takes place around the ankle when the foot is set onto the ground. Therefore, it is also helpful in this variant if it is known whether the foot and which foot is set to the ground. This can be detected with methods of gait analysis from data of the angles of the thigh and the lower leg and/or from data of an acceleration sensor at the torso. It is also possible to arrange an acceleration sensor at one or both feet or ankles. For detecting the position, velocity and acceleration of the knee, a distance of the acceleration sensor to the ankle is used. A compensation of translational accelerations of the lower leg is not required. With the data which are destined for the knee, translational accelerations of the thigh can be compensated for. With an acceleration sensor and an angular rate sensor at the thigh can, with compensation for the centrifugal acceleration and the angular acceleration of the thigh, the position, velocity and acceleration of the hip joint be calculated with the knee as a pivoting point. Then, in the same way, position, movement and acceleration of the torso can be calculated by an acceleration sensor and an angular rate sensor at the torso. The compensation can be carried out with data of the angular rate sensor as well as already known data regarding the movement of the hip joint. A place at which the acceleration sensors arranged can be used as a center of gravity approximation location. The acceleration sensor can at the same time, at least approximately, measure an acceleration at the center of gravity. With the information regarding position, movement and acceleration of the torso and/or the center of gravity approximation location, also position, velocity and acceleration of the center of gravity can be calculated. Alternatively or additionally, the acceleration at the center of gravity can be detected by an acceleration sensor in its vicinity which is provided for this, especially. Thus, the detection of a risk of a fall or a fall according to the invention is possible. Preferably, at least a part and most referred all of the mentioned sensors are integrated into garment.

The determination of the standing basis area and of at least a part of the variable portion of the relative position of standing basis area and center of gravity is analoguously also possible if no acceleration sensors or inclination sensors but angle sensors are used for both knees and hip joints. As the angle sensors do not react on acceleration is directly, a compensation for disturbing accelerations is not required. The angle sensors can preferably measure the degrees of freedom which are defined at the hip and at the knee according to the model mentioned above, wherein, for simplicity, a measurement of the rotational degree of freedom around its own axis can be relinquished. If, in case of the use of angle sensors, it shall be determined which foot is set on the ground, for example in order to determine a standing basis area of the human, this can be carried out by known methods of gait analysis, for example by an acceleration sensor at the torso and/or by evaluation of the measured positions of thigh and/or lower leg and/or by arranging an acceleration sensor at one or both feet or ankles. Preferably, the angle sensors are integrated into a leg garment. Also, systems with mixtures of angle sensors and acceleration and/or inclination sensors are possible for detection. If only angle sensors are used, the orientation of the body in relation to gravity cannot be determined therefrom. One angle sensor is connected to two parts of the body, respectively, between which the angle shall be measured, thus between lower leg and thigh as well as thigh and torso.

In order to determine connection information regarding the spatial connection between standing basis and the center of gravity and regarding the standing basis, in a similar way as described above in regard of other sensor technology, preferably the geometry of lower legs, thighs and the connection of the thighs via the hip is used in order to determine connection information of the connection between standing basis and center of gravity and the position of the feet in relation to each other. A foot approximation location can be assumed at each lower and of the lower legs. By calculation of the position of the lower legs and the thighs which are connected thereto, the position of a hip joint can be determined in relation to a foot approximation location. By the position of the torso which is known then, with the help of information regarding the position of the center of gravity in relation to the hip joint, the position of the center of gravity in regard to the foot approximation location can be detected. The hip joint can then be used as a center of gravity approximation location. In order to detect the position of the other foot, the calculation can be continued over the hip into the other thigh and to the other foot approximation location. The fixed portion in the determination of the standing basis can be calculated as described above.

A further possibility in order to determine the variable portion lies in carrying out a contactless measurement between the torso and the feet. For this, for example, ultrasound, low-energy radar or light, especially infrared light or other electromagnetic radiation can be used. Preferably, each foot will be measured from three different measurements locations such that its position in in relation to the measurements locations can be determined by the distances to the measurement locations. The measurement locations are preferably arranged at the torso or are connected thereto. The feet or footwear can, for example, be provided with a transponder or reflector, particularly a retroreflector. The transponder or reflector can be fastened at a toenail, particularly the toenail of the big toe. It is also conceivable to fix a transponder or a reflector at the ankle, for example by a belt which is laid around it. As a measurement effect, a time of flight or a degree of decrease of the intensity can be used. It is also conceivable that, for the measurement of a distance of the feet from each other, a transponder reaction is triggered at one transponder first and then at the other transponder. The measurement locations can for example be arranged at the hem of a jacket or another garment of the torso or at a belt or at a waistband. Preferably, measurement locations are present around the torso such that the feet can be detected independently of whether they are located in front of, beside or behind the body. If the variable portion of the connection information is determined in the way mentioned above, the fixed portion can be determined as described above. As a foot approximation location, a toenail or an ankle or another part of the foot which is target of the measurement can be used. One or more of the measurement locations in relation to which the measurements of the foot position take place can be considered as a center of gravity approximation location.

Preferably, in case of a contactless measurement or exclusive use of angle sensors for determination of the connection information of the connection between standing basis and center of gravity as well as the position of the feet in regard of each other, data from at least one acceleration and/or inclination sensor are used which contain information regarding the position of at least one part of the body in relation to gravity. By this, the orientation of the standing basis area in relation to the center of gravity can be associated with the direction of gravity. Preferably, an acceleration sensor or an inclination sensor is arranged close to the center of gravity of the human, preferably approximately at the height of the navel and/or in the middle of the back. If the sensor is bothering there, it is also conceivable to arrange the sensor at the side of the torso and/or in a height between navel and the hip joint or in the groin above the hip joint. The relative movements between such locations of fastening of the sensor and the center of mass of the human are not very large. The connection between those locations of fastening and the center of mass can approximately be considered as rigid. Then, it is possible to conclude by calculation from the data of the sensors at the aforementioned position at the body and with further data regarding the movement of the torso at least approximately to the acceleration and the center of mass which can be used for detection of the state of balance. It is also possible to use several acceleration sensors at the torso such that possibly existing movements between parts of the torso can be calculated and taken into account. If required, compensations as described above can be carried out. Alternatively, a further angle sensor can be employed which measures movements of the torso.

The angular position of the torso and legs around an axis in direction of gravity can be acquired by magnetic field sensors which act in the way of a compass. With the acquisition of such a rotation, the orientation of the feet can be detected which, in turn, influences the standing basis area. The accuracy of recognizing a risk of tilt fall or a tilt fall can thus be increased.

In a lea, gravity it is not present. The position at jumping off can be extrapolated by calculation using the present velocities. Additionally occurring movements can be recognized by accelerations. As these are, in general, short processes, it is for example possible to carry out a single or twofold integration of accelerations in order to get to the velocities or position, respectively. Using an angular rate sensor, angular rates can be determined directly, and angular positions can be determined by integration. In this way, also during a lea a relative position of the feet to each other can be determined, wherein the undersides of which, including the intermediate space between the feet, can be projected onto a ground plane as standing basis area. In this, it is recognizable whether a human will land in balance or whether the landing can lead to a tilt fall.

In order to improve the accuracy of recognition of a risk of tilt fall or a tilt fall, it can be taken into account that the center of mass of the human is moving if the varies his posture. From determining the connection information regarding the spatial connection between standing basis and center of gravity as well as the position of the feet to each other, the positions of thigh and lower leg are known. The mass of them can be suitably assumed and the position of the center of mass accordingly be moved by calculation. A measured or calculated direction of the acceleration at the center of gravity can further be used for simplification or can be calculated with the new coordinates of the center of gravity. As the arms have only a very low portion of the mass of the body, above all, strongly accelerated arm movements are relevant for the balance. They can be measured by acceleration sensors and be taking into account in the acceleration of the center of gravity. Furthermore, the position of the arms can then be taken into account by calculation in the position of the center of gravity. However, it is referred, for reasons of simplification of the system, to neglect the arms in regard of the recognition of a risk of tilt fall or a tilt fall. Also, the head has only a small portion of the mass of the body weight. Furthermore, it cannot be moved over large distances. Although also those movements can be detected by measurements and can be taken into account in the position of the center of gravity and the direction of the acceleration at the center of gravity, it is referred to neglect the head in regard of the recognition of a risk of tilt fall or a tilt fall.

The torso has the greatest portion of mass in the body. Variations in its form therefore have significant effects on the position of the center of gravity. Preferably, at two locations of the torso, inclination or acceleration sensors, or between the parts, one angle sensor can be fastened, wherein the two locations are arranged at different sections in vertical direction. In this way, it can be detected whether the upper part of the torso, in comparison to the lower part of the torso, is inclined to the front or to the back. Accordingly, the position of the center of gravity can be corrected by calculation. It is conceivable, to adapt also the direction of the accelerations which act at the center of gravity which can take into account the changed posture. For example, in a rotation, different distances from the pivoting point can be present. However, the same direction of acceleration at the center of gravity is preferably maintained. With one magnetic field sensor upper and lower at the torso, respectively, it is possible to detect a torsion of the torso in itself which means the shoulders in relation to the hip. It is also possible to take into account this influence on the center of gravity, particularly in combination with a bending forward or a bending backward of the upper torso, as has been described above for other variations of the posture.

Preferably, a risk of fall is recognized if, in the projection plane, the projection point is located inside the standing basis area and closer to the border of the standing basis area as a fall risk distance and/or if the projection point in the projection plane moves towards the border of the standing basis area with a relative velocity which makes expect that the border of the standing basis area is reached in less then a redefined time and/or the relative velocity is above a fall risk threshold value. In this, the fall risk distance is preferably defined normal to the border of the standing basis area. It can, however, also be defined in direction of a movement of the projection point towards the border of the standing basis area. A relative velocity towards the border of the standing basis area means that a tilting movement is present which goes along with a momentum in direction of tilting. Therefore, at a strong momentum, it can make sense to already trigger a risk of fall alert and/or further consequences if the projection point is still far away from the border of the standing basis area, as it is to be expected that it will reach in short time by the momentum. On recognition of a risk of fall, an alert can be triggered. Alternatively or additionally, on recognition of a risk of fall, a system for fall recognition can be switched to an alert mode in which, for example, one or more measurement rates are increased and/or a data processing for the further recognition of a fall is accelerated.

A state of the human is indicated as a fall in which he accelerates progressively in direction towards the ground, wherein he substantially tilts over in a tilt fall and/or collapses or slumps without strong rotation of the body at the beginning of the fall in a vertical fall. Mixed forms of the tilt fall and the vertical fall are possible. A fall can be divided into two phases, namely the beginning of a fall during which the falls who can be prevented by a catching reaction and a ground fall in which is to be expected with a high probability that the human falls to the ground.

Preferably, a tilt fall is recognized, which may be a pure tilt fall or a part of a mixed fall if the projection point in the projection plane is located outside of the standing basis area wherein, preferably, a beginning fall is recognized if the distance of the projection point from the border of the standing basis areas less than a ground fall threshold distance and/or the relative velocity of the projection point in the projection plane is less than a ground fall relative velocity and/or a relative acceleration in the projection line is less than a ground fall relative acceleration. If the momentum in the tilting movement is still so small that sufficient time is left for the human in order to carry out a catching reaction and/or the momentum energy is still so small that it can be caught by the catching reaction, the human still can prevent the ground fall by own forces. Such a catching reaction preferably concerns a reaction with the feet in order to bring the standing basis area under the projection point again. Alternatively or additionally, the reaction can also be carried out with the hands or another section of the body with the help of fixed objects such as a railing, a lamppost, a piece of furniture, a wall or the like. If a catching reaction has been successful and the projecting point moves back to the standing basis area, or the standing basis area moves under the projection point, respectively, the recognition of a fall can be canceled.

The recognition of a fall can also be canceled if the velocity or acceleration of the projection point in the standing basis area is lower than a redefined velocity or a redefined acceleration, respectively, which is to be expected in a fall.

If a successful catching reaction is expectedly not possible any more, a ground fall can be recognized, namely, if the distance of the projection point from the border of the standing basis area is greater than a ground fall relative velocity and/or the relative velocity of the projection point is greater than a ground fall acceleration, or a ground fall can be detected from a velocity at the torso, the wrist or at the head which is greater than the respective threshold velocity. All these considerations take place in the projection plane. Preferably, the ground fall distance and/or the ground fall relative velocity are set such that in excess of the redefined distance or the redefined velocity, it is not to be expected any more that a reaction of the human can still stop the fall significantly.

The ground fall distance and/or the ground fall relative velocity and/or the ground fall relative acceleration can be calculated, alternatively or additionally, from a critical velocity and/or energy of the tilting process and/or from an expected critical rest time until impact on the ground plane. It is conceivable to determine the ground fall relative velocity and/or the ground fall relative acceleration from the distance of the projection point from the border of the standing basis area, as in a common tilting process, relative velocities and relative acceleration increase with the distance, such that it can be assumed in approximation that in a certain distance a certain velocity or acceleration, respectively, is present. In a calculation of the ground fall distance, an already present velocity of the projection point in the standing basis area be taken into account when defining a redefined velocity. The method that is described in this paragraph is independent of whether a tilt fall, a vertical fall or a mixed fall this present.

In a situation in which the projection point is located outside of the standing basis area and the human supports himself with another part of the body as the feet or holds on, a ground fall can be recognized if a velocity or an acceleration of the projection point away from the border of the standing basis area or a velocity or acceleration in direction to the ground plane occurs which is greater than a vertical fall velocity and/or a vertical fall acceleration and/or a vertical ground fall velocity and/or a vertical ground fall acceleration, wherein, particularly, the vertical fall velocity and/or the vertical fall acceleration and/or the vertical ground fall velocity and/or the vertical ground fall acceleration is determined in dependency of the distance of the projection point from the border of the standing basis area. In this way can be determined whether a human which supports himself outside of his balance or holds on, loses hold and falls, or is at the beginning of fall and is threatened to fall.

In a vertical fall by slumping of a human, the support of the center of gravity by at least one leg fails. Such a fall can, for example, be a consequence of an unconsciousness. Often, not both legs fail at the same time, but at first one, which leads to a movement of the projection point out of the standing basis. Then, tilting over takes place. If the cause is unconsciousness, in a normal case, no catching reaction is possible any more. In this case, the strategy for recognizing a fall can be used as described above. If the slumping takes place essentially downwards, it can happen that the projection point does not move out of the standing basis area in this.

A fall without a great portion of tilting can be recognized in that from the beginning of the fall on, velocity and/or acceleration of the torso towards the ground plane is present. When exceeding a vertical fall velocity or a vertical fall acceleration towards the ground, a beginning fall can be recognized. When exceeding a vertical ground fall velocity towards the ground which is greater than the vertical fall velocity, a ground fall can be recognized. Additionally to this recognition, a monitoring of the position of the projection point in relation to the border of the standing basis area can be carried out in the projection plane.

When a human is sitting down, he moves out of his balance as the feet in very many cases have to stand in front of the seat, whereas the center of gravity is positioned above the seat. In this, the protection point moves out of the standing basis area. In order not to hurt themselves when sitting down, humans limit the impact seed onto the seat or generate a braking acceleration against the falling acceleration. Furthermore, they often hold on to this purpose. In order to prevent injuries and in order to distinguish real falls from sitting down, a threshold for the velocity or a time period of an acceleration of the torso downwards can be used in order to determine that, when exceeded, an advanced fall is taking place. In this, it can be taken into account that the impact plane is located in the height of the seat, typically.

When detecting a risk of fall or a fall, it is possible, to take into account additional data such as data from an angular rate sensor, which measures a tilting rotation velocity of a torso of the human, or data from an acceleration sensor which measures the acceleration in direction towards the ground, or data from a system that calculates a position of the center of gravity in relation to a ground plane. From these or further data, for example a fall velocity of the center of gravity in direction to the ground can be derived. This is advantageous, as there are not only falls with a tilt movement, but also falls by slumping or falls to the ground in which at least a part of the fall can take place without the projection point leaving the standing basis area, in the projection plane. Then, different types of falls, their mixed forms and border regions between them can be recognized better.

To this purpose, it is proposed to correct the position or velocity or acceleration of the projection point in relation to the border of the standing basis area in the projection plane for recognizing a tilting movement and/or threshold values with which the aforementioned values are compared on basis of values of a velocity or an acceleration of the center of gravity and/or of a head and/or of the wrists in direction to the ground. For example, a relative velocity of the projection point in relation to the border of the standing basis area can be increased for the purpose of comparison with a threshold, if it is determined that, in excess of a tilting movement, a downward velocity of the center of gravity is present.

Particularly in the determination whether it is a ground fall in which a successful catching reaction cannot be expected any more, the correction can, for example, be carried out in a way to take into account whether the impact is expected earlier and/or how great the impact velocity or impact energy is. An influencing factor which depends on a velocity of the center of gravity downloads and/or an influence factor which is depending on a distance of the projection point from the border of the standing basis area in the projection plane can be determined empirically. In this way, it can be taken into account that a fall can be partially a tilt fall and partially a vertical fall. The assessment whether a beginning fall is present or a ground fall is to be expected, is thereby improved. Alternatively or additionally, when recognizing a vertical fall by slumping or the like, also a downward velocity of the center of gravity or of the head or of the wrists can be corrected by the position or the velocity or the acceleration of the projection point in relation to the border of the standing basis area in the projection plane. The same advantage results.

For example, a velocity or an acceleration of the center of gravity downwards can be used as a further influence on the determination of a ground fall distance, the ground fall relative velocity or a ground fall acceleration. These values, as well as a fall risk threshold value, can be set in dependence on a situation in which the human is, for example standing, walking, jogging/running, jumping, ascending stairs, descending stairs, sitting down, sitting, standing up from sitting, laying down, lying, standing up from lying, leaning on, squatting, carrying something or picking up into the arms, hugging somebody, holding on, supporting oneself, going with a rolling frame, going with a stick, dancing, washing, going to the toilet, kneeling, going arms and arms, bicycling, unknown context. Special situations, such as certain types of sorts in which a similar situation of balancing is present such as in skiing or snowboarding, skating or rollerskating, are also conceivable. Also, a narrow standing-on-ground area of, for example, of skating shoes or rollerskating shoes is valid as a standing basis area or part thereof. For drivers of two-wheeled vehicles, the method according to the invention can also be applied, wherein the standing basis area is defined by a standing-on-ground area defined by the area at which the wheels are standing on the ground and their intermediate area. The method can also be applied when walking with a rolling frame. For determining the relative position between the standing basis area of a two-wheeled vehicle and a center of gravity which is to be considered as the center of gravity of the vehicle including the human who drives it, it can be required to evaluate one or more sensors at the two-wheeled vehicle, for example an acceleration or inclination sensor. It is, however, also possible to use a fixed portion between the center of gravity and the standing basis area. The method can also applied for sportsmen who carry out board-based sorts such as snowboarding, skateboarding or wind skating. Then, the position of the feet is, in general, extensively fixed such that the standing basis is constant. The method can be used under this condition.

In all cases, accelerations from driving a curve, breaking and acceleration operations can be taken into account, additionally. This is also true for humans without sorting devices. The method can also be combined with a method for collision warning which also can trigger the same measures which are triggered at a ground fall, or a part thereof.

If a ground fall is recognized, a signal can be generated which initiates protection measures against an impact. This can, for example, be unfolding of expandable airbags.

Alternatively or additionally, a signal can be generated which introduces emergency measures for the fallen human, for example an alert at an aiding organization.

A vertical fall recognition as described above is proposed as an independent invention. The variable portion can be a determined as described above.

In a further embodiment, it is proposed to carry out the fall recognition in a context-based way. This means that from the data which are present from the person, a context is determined which the person currently is subject of. A list of possible occupations can be found six paragraphs further above. The list can be replenished by further activities of daily life. As, for fall recognition in different contexts, different criteria for recognizing the risk of fall, the beginning of a fall and the ground fall are appropriate, the context-based fall recognition represents a substantial contribution for improving the invention. For recognizing the context, pattern recognition can be used. For this, data can be used which originate from the person and which represent his current or recent body posture. particularly, typical sequences of such body postures can be used as a pattern for a certain context. corresponding methods for assessing the similarity of a measured sequence of body postures regarding an ideal sequence of postures which belong to a certain context are common knowledge from pattern recognition.

In a further embodiment, it is proposed to span a state space by movement data in order to classify the current state of the human into a class by boundaries in state space. The boundaries in the state space represent boundaries of classes. Coordinates of such a space state can, for example, be:
1. the position of the projection point in relation to the standing basis, particularly the distance of the projection point to the border of the standing basis (two-dimensional),
2. the velocity of the projection point in regard of the border of the standing basis (two-dimensional),
3. the height of the center of gravity above the standing basis (one-dimensional),
4. the vertical velocity of the center of gravity of (one-dimensional),
5. the vertical acceleration of the center of gravity (one-dimensional),
6. the vertical velocity at the hip (one-dimensional),
7. the relative angle between each of the thighs and the torso (2 times two-dimensional as the hip joint has two dimensions of freedom) and
8. the angle between the torso and the gravity (one-dimensional).

All dimensions or a part of them can be uses. Preferably, at least the first three of the above-mentioned dimensions are used.

By definition of boundaries in the state space, different classes can be defined. Classes can for example be: no risk of fall—risk of fall—beginning fall—fall cannot be prevented any more/ground fall—impact. The boundaries can be determined depending on the context. For this, depending on the context, criteria can be used, for example, for the boundary between risk of fall and fall, the vertical velocity at the hip in connection with the height above the standing basis can be used. Another example for distinguishing risk of fall and fall could be that the projection point is located outside of the standing basis and has a great distance from the border of the standing basis. Also, the definition of the boundaries can be influenced by which kind of persons concerned, for example, whether he is frail or disabled or athletic or average. By this course of action, the recognition whether a certain class is present, is supported by more data which can make the recognition more reliable.

In a further aspect of the invention, an apparatus for recognizing of a risk of tilt fall is established, wherein the apparatus is configured to carry out one of the methods according to one of the methods described above.

In a further aspect of the invention, a fall protection garment is established with an apparatus which comprises an apparatus which is configured to carry out one of the methods described above.

Preferably, the apparatus is integrated in to a garment for the torso and into a garment for the lower part of the body. Preferably, into each of the garments, electronics are integrated. Preferably, between the two garments, a radio connection is established. Alternatively or additionally, an operative system can be integrated into the garment for the lower part of the body, wherein all required sensors are integrated therein, and the position of the center of gravity is inferred to by calculation. For the lower part of the body, also a harness can be provided at which sensors and preferably also electronics are arranged. Preferably, a sensor is arranged close to the coccyx in order to measure the angle in regard to gravity. Alternatively or additionally, an operative system can be integrated into the garment for the torso wherein the position of the feet is detected by a remote measurement, for example by a radar or ultrasonic distance measurement with an electromagnetic sender at it, and at least one transponder can be arranged at at least one foot which can be glued to it, particularly to a toenail, and/or foot belt and/or sock and/or shoe.

In a further embodiment of the apparatus, it comprises an alert device. This can be activated if a risk of fall is recognized. Alternatively or additionally, the alert device can be activated if a beginning of a fall is recognized. This alert can be different from the alert of a risk of falls. As an alert, for example, an acoustical or optical signal, a vibration which is conveyed to the body of the human, a thermal and/or a mechanical stimulus of the skin of the human and/or an electrical simulation of the human, for example by electrodes on the skin, and/or a chemical stimulation of the human, for example in the nose or at the skin. Purpose of the frame can be to draw attention to of the human to a threatening fall or a fall and, possibly, avoid a ground fall by a reaction of the human. Furthermore, other humans can become attentive and help the human who is threatened by a fall or who is falling. In a referred variant, such an apparatus with an alert function is carried out as underwear. The alert function can, however, also be integrated into garment for the torso. An integration into everyday garment is referred.

The figures in the appendix show, as an example only, embodiments of the invention. It is shown in:

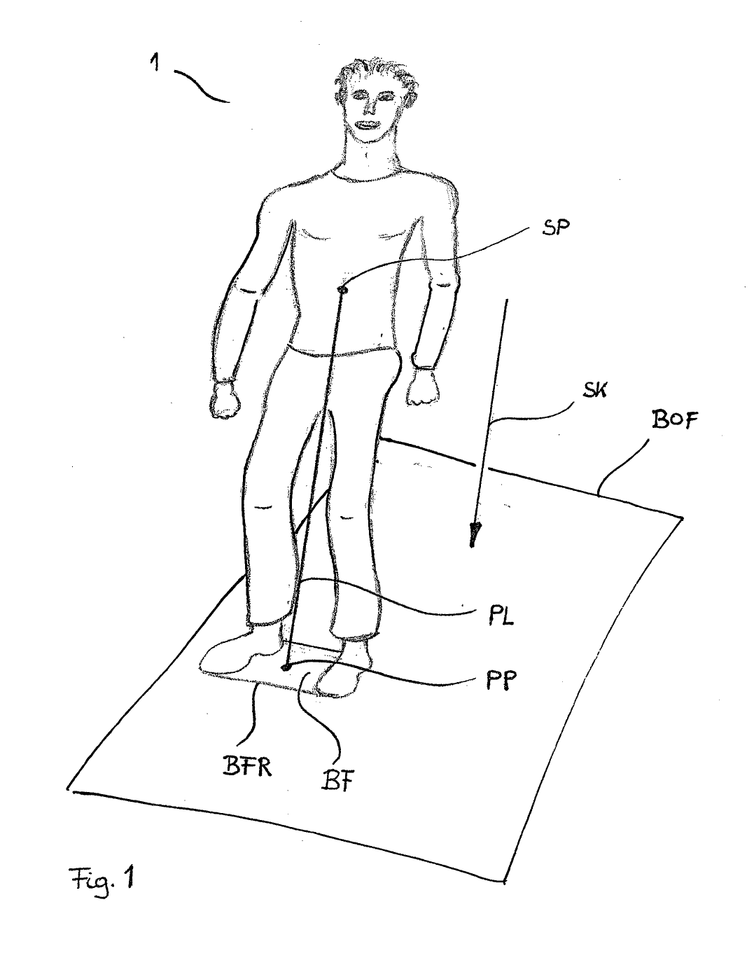

FIG. 1 a human in a perspective view obliquely from the front during standing and in his balance, in which the center of gravity is above the standing basis area,

FIG. 2 a human in a perspective view obliquely from the front in a beginning fall to the backside out of this balance, wherein the projection point is not in the standing basis area,

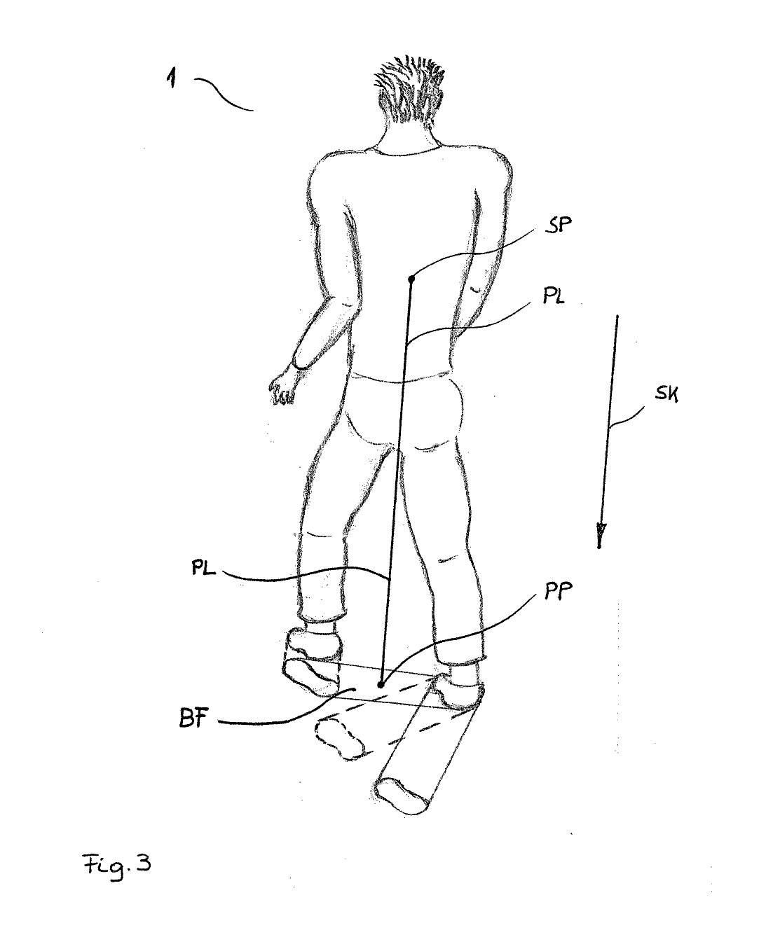

FIG. 3 a human in a perspective view obliquely from the backside during walking in his balance, wherein the center of gravity is located above the standing basis area,

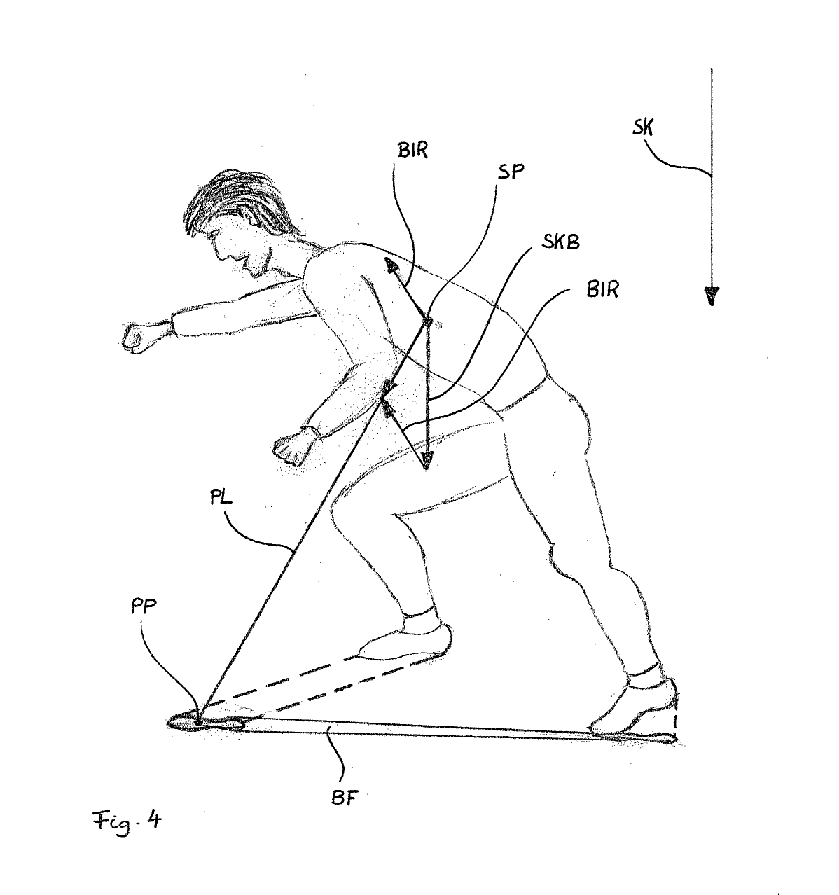

FIG. 4 a human in a view from the side at the beginning of a sprint wherein he is in a dynamic balance which is stable, and

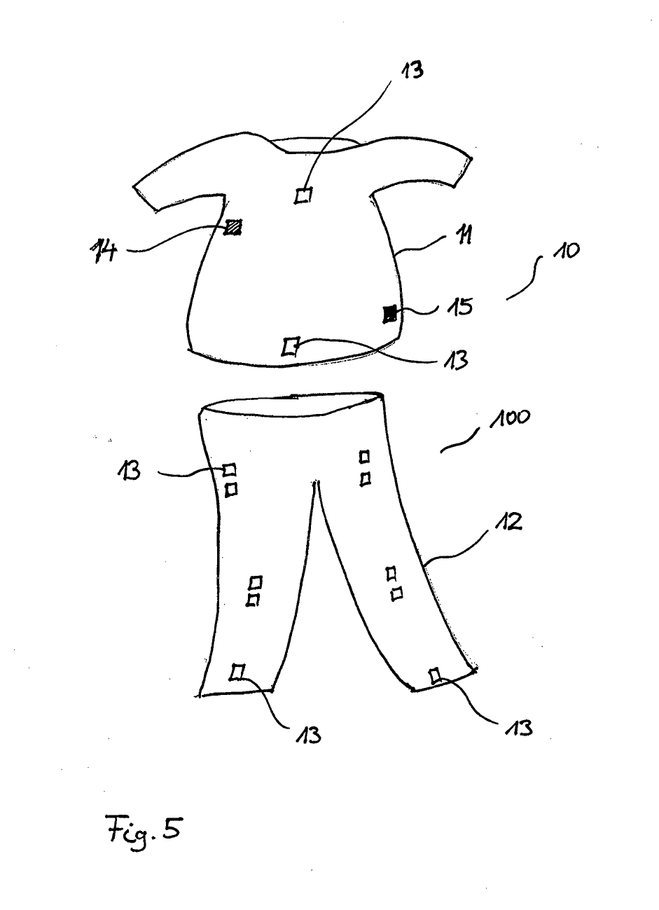

FIG. 5 schematically a perspective view of an underwear with an apparatus according to the invention including an alert device.

FIG. 1 schematically shows a human 1 during standing who is in his balance. The center of gravity is located in the torso somewhat above the height of the navel. The human 1 stands on a ground plane which is not explicitly depicted, in which a standing basis area BF is located. The standing basis area BF has a border of the standing basis area BFR. The border of the standing basis area extends at the outsides of the feet, at the front sides of the feet and at the heels. Between the feet, an area is located that is also part of the standing basis area BF. This area is limited by a line between the foot tips and a line between the ends of the heels which also are a part of the border of the standing basis area and by the inner sides of the feet. The center of gravity is located in direction of gravity SK above the standing basis area BF. As no accelerations in space are present, the gravity is the only force on the human 1. Beginning from the center of gravity SP, a projection line PL is running in direction of gravity SK. At the intersection point of the projection line PL with the standing basis area, the projection point PP is located. Its position inside of the standing basis area indicates that the human 1 is in balance.

FIG. 2 schematically shows the same human in a fall to the back side. In the whole, FIG. 2 is similar to FIG. 1. The same features are denoted with the same reference signs and will not be described separately once again. In difference to FIG. 1, the human 1 in FIG. 2 has significant layback and is endangered to fall backwards onto the ground plane in which the standing basis area BF is located.

Accordingly, the projection point is located in direction of the back of the human 1, far outside of the standing basis area BF. In order to catch the fall, the human 1 has lifted one foot in the intention to move it backwards. The standing basis area BF is therefore defined by one foot which is set on the ground and by the projection of a lifted foot into the ground plane. The projection is represented by two dotted lines between the left foot of the person and the standing basis area BF. The projection has been carried out in direction of gravity SK. Also, other projection directions are conceivable. For example, the direction of gravity can be different from the acceleration of center of gravity if the human 1 has been shoved before and has additionally been accelerated in space by this. If it is successful to move the foot to the backside very fast, it could the possible to move the standing basis area BF backwards so far that the projection point PP is located in the standing basis area BF again. Then, the fall would have been caught and the human 1 would not fall onto the ground plane. In this consideration, it is to be taken into account that also the projection point is moving further to the back as the tilting over is accompanied by a relocation of the center of gravity backwards-down. Moreover, a backward acceleration of the center of gravity takes place. This has the consequence that the direction of the acceleration at the center of gravity which results from geometric addition of gravity acceleration and the acceleration of the center of gravity in space also is moved further backwards, by which the projection point moves backwards even further. Thus, in the course of a tilt fall, it becomes increasingly more difficult to still catch it. Therefore, a limiting distance can be determined from which on the tilt fall is recognized as a ground fall and rescue operations such as the opening of airbags can be triggered.

In FIG. 2, further, a connection line VL between the center of gravity and the border of the standing basis area is drawn. With its help, it can be determined in which phase the fall is. The decisive criterion for the progress of the fall is the angle between the connection line VL and the direction of acceleration of the center of gravity, along which the projection line PL is running in FIG. 2. The bigger the angle is, the further the fall has proceeded, if the angle lies in such a way that it opens away from the standing basis area BF. The location of the border of the standing basis area BFR which forms the foot point at the lower end of the connection line VL is preferably chosen starting from the middle of the standing basis area BF in the direction in which the center of gravity SP moves in the coordinates of the standing basis area. As the standing basis area can move by balancing movements with the foot, it can be required to calculate the foot point of the connection line VL and the connection line VL itself multiple times. It is, however, not required to calculate the projection line or the protection point. It is sufficient if the direction of the acceleration at the center of gravity is known.

FIG. 3 shows the human 1 from the backside when walking. Regarding the shown features, FIG. 1 is similar to FIG. 2. The same features are denoted with the same reference signs and will not be described separately once again. Reference is made to the description of FIG. 1. When walking, the human is in a dynamic balance in which, in each step, the beginning of a tilt fall and a catching in the next step takes place. If one foot is lifted, the human tilts to the front in the ankle of the foot which is set on the ground. The lifted foot is lead in such a way that, if it is set on the ground, it catches the tilting to the foot again. In this way, it becomes the foot that is set on the ground, whereby the roles of the feet alternate in the same process.

The part of the standing basis area which belongs to a lifted foot is for example determined for a lifted foot by projecting it in direction of gravity onto the ground plane where the projection forms are part of the standing basis area BF. The accelerations of the center of gravity in space are small in a steady gait such that the direction of the projection line does not differ much from the direction of gravity SK.

FIG. 4 shows the human 1 from the side when starting a sprint. Regarding the shown features, FIG. 2 is similar to FIG. 1. The same features are denoted with the same reference signs and will not be described separately once again. Reference is made to the description of FIG. 1. The backward foot is lifted and provides for acceleration of the human 1 by exerting backward force onto the ground. At the same time, the human moves into an upright position. An acceleration BIR of the center of gravity in space towards front-u results. A triangle of vectors is drawn which represents the geometric addition of the acceleration BIR of the center of gravity in space with the gravity SK. The acceleration BIR of the center of gravity is therefore strongly directed to the front. correspondingly, a projection line L results which is strongly inclined to the front. As the human stretches his lifted foot far to the front, a very much elongated standing basis area BF results in which the projection point PP is being located. Therefore, here, no risk of fall is present.

FIG. 5 shows a schematic representation of a garment 10 in form of underwear with an apparatus 100 for recognizing a fall. The underwear comprises an upper part 11 and a lower part 12 with long legs. The apparatus 100 for recognizing a fall comprises twelve sensors 13 which can one-dimensionally measure an acceleration and an angular rate in three dimensions. These sensors 13 are arranged at each hem of the legs of the lower part 12, above and below the knee as well as above and below the hip. Two further sensors 13 are arranged at the upper part 11, one of which is arranged at the lower end and one in the middle of the back. Further, the garment 10 comprises an alert device 14 which is part of the apparatus 100. Further, the apparatus 100 comprises a calculation unit, which is arranged in the upper part 11.

### LIST OF REFERENCE SIGNS

1 Human
BIR Acceleration in Space
BF Standing Basis Area
BOF Ground Plane
BFR Border of the Standing Basis Area
PL projection Line
PP projection point
SK Direction of Gravity
SKB Acceleration of Gravity
SP Center of Gravity
VL Connection Line

Embodiments according to following paragraphs are proposed.

Paragraph 1

A Method for detecting a tilt fall or a risk of a tilt fall, proposed with the following steps: detecting a center of gravity position of the center of gravity of a human which represents an actual center of mass of the human or an approximation thereto, detecting a position of a standing basis of the human or an approximation thereto, detecting connection information regarding a spatial connection between the center of gravity and the standing basis, comprising the steps
detecting an angular position of at least one lower leg of a leg in relation to the associated thigh and an angular position of the same thigh to the torso,
calculating a relative position of the ankle or at least a part of the foot or a footwear of the leg to the center of gravity as connection information, wherein angular positions between lower leg and thigh as well as between thigh and torso as well as lengths of the lower leg and the thigh as well as a relative position between the hip joint of the leg and the center of gravity are used,
deriving a tilt fall or a risk of a tilt fall from the grade of correctness that the center of gravity and the standing basis are located in direction of the acceleration of gravity in relation to each other.

Paragraph 2

Method according to paragraph 1, characterized in that a vertical fall is recognized if a velocity or an acceleration of a torso and/or of a center of gravity of the human in direction to the standing basis area is recognized of which the amount is greater than a threshold value, wherein, particularly, a beginning vertical fall is recognized if an excess of a vertical fall velocity and/or a vertical fall acceleration is recognized and/or a vertical ground fall is recognized if an excess of a vertical ground fall velocity and/or a vertical ground fall acceleration is recognized.

Paragraph 3

Method according to paragraph 2, in which in recognition of a mixed fall of a tilting fall and a vertical fall, while recognizing of a beginning fall, a vertical fall velocity and/or a vertical fall acceleration and/or a vertical ground fall velocity and/or a vertical ground fall acceleration, the position or velocity or acceleration of the projection point in relation to the border of the standing basis area in the projection plane for recognition of a tilt fall and/or thresholds with which said values are compared for recognition of a tilt fall, are corrected on basis of values of a velocity or an acceleration of the center of gravity in direction to the ground.

Paragraph 4

Method according to one of the preceding paragraphs, characterized in that the standing basis is detected as a standing basis area, wherein the standing basis (BF) comprises:

if a first foot or a foot wear of the first foot is set onto a ground plane (BOF), an area of the first foot or of a footwear of the first foot, particularly an underside thereof, as first foot standing basis area (FBF1) and,

if the first foot or a footwear of the first foot is lifted from the ground plane (BOF), a projection of the first foot or a footwear of the first foot, particularly an underside thereof, into the ground plane (BOF) as first foot standing basis area (FBF1), and,

if a second foot or a footwear of the second foot is set onto the ground plane (BOF), an area of the second foot or a footwear of the second foot, particularly an underside thereof, as second foot standing basis area (FBF2) and

if a second foot or a footwear of the second foot is lifted from the ground plane (BOF), a projection of an area of the second foot or a footwear of the second foot, particularly an underside thereof, into the ground plane (BOF) as a second foot standing basis area (FBF2), and

particularly an intermediate standing basis area (ZBF) between the first foot standing basis area (FBF1) and the second foot standing basis area (FBF2).

Paragraph 5

Method according to one of the paragraphs 3 or 4, characterized in that a projection of the first or second foot or a footwear of the first or second foot or an underside thereof into the ground plane (BOF) as a part of the basis area (BF) is determined in direction of gravity or in direction of movement of the foot.

Paragraph 6

Method according to one of the preceding paragraphs, characterized in that the acceleration of the center of gravity comprises an acceleration of gravity at the center of gravity and, particularly, additionally comprises an acceleration at the center of gravity in space and/or a centrifugal acceleration at the center of gravity.

Paragraph 7

Method according to one of the paragraphs 3 to 6, characterized by the following steps:
projecting a center of gravity of the human in direction of the acceleration of the center of gravity into a standing basis area of the human as a projection plane or projecting the standing basis area or at least a part of the border of the standing basis area in opposite direction to the acceleration of the center of gravity into a protection plane in which the center of gravity is located in which is arranged in an angle to the acceleration of the center of gravity which, particularly, is 90°,
particularly, detecting of a reference location of a border of the standing basis area in the projection plane, and
deriving a risk of a tilt fall or a tilt fall from the position of the projection point in relation to the border of the standing this area and, particularly, to the reference location,
wherein, particularly, a risk of a tilt fall is recognized, if in the projection plane, the projection point is located inside the standing basis area and
the projection point is located closer to the border of the standing basis area, and, particularly, to the reference location, as a fall risk distance and/or
the projection point moves towards the border of the standing basis area or the reference location on the border of the standing basis area or the reference location, respectively, which makes expect that the border of the standing basis area or the reference location is reached in less than a redefined time and/or the relative velocity is above a fall risk threshold value, and/or
wherein, particularly, a fall risk is recognized if, in the projection plane, the projection point is located outside of the standing basis area, wherein, preferably,
a beginning of a tilt fall is recognized if
the distance of the projection point from the border of the standing basis area or the reference location is smaller than a ground fall threshold distance, and/or
a relative velocity is less than a ground fall relative velocity, and/or
a relative acceleration is less than a ground fall relative acceleration, and/or
a ground fall is recognized if
the distance of the projection point from the border of the standing basis area or the reference location is greater than the ground fall threshold distance and/or
the relative velocity of the protection point is greater than the ground fall relative velocity and/or
a relative acceleration is greater than a ground fall relative acceleration.

Paragraph 8

Method according to one of the paragraphs 3 to 6, characterized by the following steps:
detecting a deviation of directions between
a theoretical connection line (VL) from the center of gravity (S) to a reference location at the border of the standing basis area (BF) and
the direction of an acceleration of the center of gravity (GK),
deriving a risk of tilt fall or a tilt fall from the deviation of directions,
wherein, particularly, the risk of a tilt fall is recognized, if the direction of the acceleration of the center of gravity is, starting from the center of gravity (S) or from the projection point (PP), directed towards the standing basis area in comparison to the direction of the connection line, and
the deviation of directions is less than a fall risk direction deviation, and/or
the deviation of directions decreases with a change rate which makes expect that that it becomes at least approximately zero in less than a redefined time and/or the change rate is above a fall risk threshold rate, and/or

wherein, particularly, a tilt fall is recognized if the direction of the acceleration of the center of gravity is, starting from the center of gravity (S) or from the protection point ( ), is directed away from the standing basis area in comparison to the connection line, wherein, preferably,
a beginning of a tilt for this recognized if
the deviation of directions is less than a ground fall threshold deviation and/or
the change rate of the deviation of directions is less than a ground fall change rate, and/or
a derivation in respect of time of the change rate is less than a second ground fall change rate, and/or
a ground fall is recognized if
a deviation of directions is greater than a ground fall threshold deviation and/or
the derivation in respect of time of the change rate is greater than a second ground fall change rate.

Paragraph 9

Method according to one of the preceding paragraphs, characterized in that the relative position of the standing basis to the center of gravity is detected from a changing variable portion and, optionally, additionally from a non-variable fixed portion, wherein, particularly, the variable portion of the relative position between a foot approximation location close to a foot or at the foot and
a center of gravity approximation location at or close to the torso of the human or at or close to the center of gravity of the human
influences the detection of the connection information, and

particularly, the non-variable fixed portion of the relative position of the foot approximation location and an associated foot standing basis area (FBF1, FBF2) and/or between the center of gravity approximation location and the center of gravity influences the detection of the connection information.

Paragraph 10

Method according to one of the preceding paragraphs, characterized in that the variable portion is determined by
using data from one or more sensors which measure the bending of a knee directly or indirectly,
using data from one or more sensors which measure the position of a hip joint directly or indirectly,
using the length of a lower leg and a thigh and
detecting a variable portion between the torso and the lower leg of the human,
wherein, particularly for indirect measurement, an inclination sensor for measurement of the inclination in relation to gravity and/or an acceleration sensor with a static measurement function is used which are, preferably, integrated into a garment, and/or, preferably, interfering accelerations from a rotation of a part of the body with which the inclination sensors or acceleration sensors are connected mechanically for measurement, are compensated for by help of an angular rate sensor and/or, for direct measurement, angle sensors are used which are, for measurement of an angle of parts of the body in relation to each other, connected with these parts of the body and which are, particularly, integrated into garment.

Paragraph 11

Method according to one of the preceding paragraphs, characterized in that, as reaction on a risk of fall, a beginning fall and/or a ground fall, an alert is triggered in order to make the human attentive for the threatening fall or the fall, wherein the alert is particularly an acoustic and/or optical signal, a vibration which is transferred to the body of the human, a thermal and/or mechanical stimulus and/or an electrical stimulus of the human, for example by electrodes on the skin, and/or a chemical stimulus of the human, for example in the nose or on the skin.

Paragraph 12

Apparatus (100) for recognizing a risk of a tilt fall or a tilt fall, wherein the apparatus (100) is configured for carrying out of a method according to one or more of the preceding claims.

Paragraph 13

Fall projection garment (10) with an apparatus according to paragraph 12, wherein, particularly, the fall projection garment (10) has the form of an underwear (11, 12), wherein, particularly, the underwear (11, 12) is realized tightfitting at least at a location at which a sensor is arranged.

1. Method for detecting a tilt fall or a risk of a tilt fall, proposed with the following steps:
detecting a center of gravity position of the center of gravity of a human which represents an actual center of mass of the human or an approximation thereto,
detecting a position of a standing basis of the human or an approximation thereto,
detecting connection information regarding a spatial connection between the center of gravity and the standing basis, comprising the steps
detecting an angular position of at least one lower leg of a leg in relation to the associated thigh and an angular position of the same thigh to the torso,
calculating a relative position of the ankle or at least a part of the foot or a footwear of the leg to the center of gravity as connection information, wherein angular positions between lower leg and thigh as well as between thigh and torso as well as lengths of the lower leg and the thigh as well as a relative position between the hip joint of the leg and the center of gravity are used,
deriving a tilt fall or a risk of a tilt fall from the grade of correctness that the center of gravity and the standing basis are located in direction of the acceleration of gravity in relation to each other.

2. Method according to claim 1, characterized in that a vertical fall is recognized if a velocity or an acceleration of a torso and/or of a center of gravity of the human in direction to the standing basis area is recognized of which the amount is greater than a threshold value, wherein, particularly, a beginning vertical fall is recognized if an excess of a vertical fall velocity and/or a vertical fall acceleration is recognized and/or a vertical ground fall is recognized if an excess of a vertical ground fall velocity and/or a vertical ground fall acceleration is recognized.

3. Method according to claim 2, in which in recognition of a mixed fall of a tilting fall and a vertical fall, while recognizing of a beginning fall, a vertical fall velocity and/or a vertical fall acceleration and/or a vertical ground fall velocity and/or a vertical ground fall acceleration, the position or velocity or acceleration of the projection point in relation to the border of the standing basis area in the projection plane for recognition of a tilt fall and/or thresholds with which said values are compared for recognition of a tilt fall, are corrected on basis of values of a velocity or an acceleration of the center of gravity in direction to the ground.

4. Method according to claim 1, characterized in that the standing basis is detected as a standing basis area, wherein the standing basis (BF) comprises:
if a first foot or a foot wear of the first foot is set onto a ground plane (BOF), an area of the first foot or of a footwear of the first foot, particularly an underside thereof, as first foot standing basis area (FBF1) and,
if the first foot or a footwear of the first foot is lifted from the ground plane (BOF), a projection of the first foot or a footwear of the first foot, particularly an underside thereof, into the ground plane (BOF) as first foot standing basis area (FBF1), and,
if a second foot or a footwear of the second foot is set onto the ground plane (BOF), an area of the second foot or a footwear of the second foot, particularly an underside thereof, as second foot standing basis area (FBF2) and
if a second foot or a footwear of the second foot is lifted from the ground plane (BOF), a projection of an area of the second foot or a footwear of the second foot, particularly an underside thereof, into the ground plane (BOF) as a second foot standing basis area (FBF2), and
particularly an intermediate standing basis area (ZBF) between the first foot standing basis area (FBF1) and the second foot standing basis area (FBF2).

5. Method according to claim 3, characterized in that a projection of the first or second foot or a footwear of the first or second foot or an underside thereof into the ground plane (BOF) as a part of the basis area (BF) is determined in direction of gravity or in direction of movement of the foot.

6. Method according to claim 1, characterized in that the acceleration of the center of gravity comprises an acceleration of gravity at the center of gravity and, particularly, additionally comprises an acceleration at the center of gravity in space and/or a centrifugal acceleration at the center of gravity.

7. Method according to claim 3, characterized by the following steps:
projecting a center of gravity of the human in direction of the acceleration of the center of gravity into a standing basis area of the human as a projection plane or projecting the standing basis area or at least a part of the border of the standing basis area in opposite direction to the acceleration of the center of gravity into a protection plane in which the center of gravity is located in which is arranged in an angle to the acceleration of the center of gravity which, particularly, is 90°,
particularly, detecting of a reference location of a border of the standing basis area in the projection plane, and
deriving a risk of a tilt fall or a tilt fall from the position of the projection point in relation to the border of the standing this area and, particularly, to the reference location,
wherein, particularly, a risk of a tilt fall is recognized, if in the projection plane, the projection point is located inside the standing basis area and
the projection point is located closer to the border of the standing basis area, and, particularly, to the reference location, as a fall risk distance and/or
the projection point moves towards the border of the standing basis area or the reference location on the border of the standing basis area or the reference location, respectively, which makes expect that the border of the standing basis area or the reference location is reached in less than a redefined time and/or the relative velocity is above a fall risk threshold value, and/or
wherein, particularly, a fall risk is recognized if, in the projection plane, the projection point is located outside of the standing basis area, wherein, preferably,
a beginning of a tilt fall is recognized if
the distance of the projection point from the border of the standing basis area or the reference location is smaller than a ground fall threshold distance, and/or
a relative velocity is less than a ground fall relative velocity, and/or
a relative acceleration is less than a ground fall relative acceleration, and/or
a ground fall is recognized if
the distance of the projection point from the border of the standing basis area or the reference location is greater than the ground fall threshold distance and/or
the relative velocity of the protection point is greater than the ground fall relative velocity and/or
a relative acceleration is greater than a ground fall relative acceleration.

8. Method according to claim 3, characterized by the following steps:
detecting a deviation of directions between
a theoretical connection line (VL) from the center of gravity (S) to a reference location at the border of the standing basis area (BF) and
the direction of an acceleration of the center of gravity (GK),
deriving a risk of tilt fall or a tilt fall from the deviation of directions,
wherein, particularly, the risk of a tilt fall is recognized, if the direction of the acceleration of the center of gravity is, starting from the center of gravity (S) or from the projection point (PP), directed towards the standing basis area in comparison to the direction of the connection line, and
the deviation of directions is less than a fall risk direction deviation, and/or
the deviation of directions decreases with a change rate which makes expect that that it becomes at least approximately zero in less than a redefined time and/or the change rate is above a fall risk threshold rate, and/or
wherein, particularly, a tilt fall is recognized if the direction of the acceleration of the center of gravity is, starting from the center of gravity (S) or from the protection point (PP), is directed away from the standing basis area in comparison to the connection line, wherein, preferably,
a beginning of a tilt for this recognized if
the deviation of directions is less than a ground fall threshold deviation and/or
the change rate of the deviation of directions is less than a ground fall change rate, and/or
a derivation in respect of time of the change rate is less than a second ground fall change rate, and/or
a ground fall is recognized if
a deviation of directions is greater than a ground fall threshold deviation and/or
the derivation in respect of time of the change rate is greater than a second ground fall change rate.

9. Method according to claim 1, characterized in that the relative position of the standing basis to the center of gravity is detected from a changing variable portion and, optionally, additionally from a non-variable fixed portion, wherein, particularly, the variable portion of the relative position between a foot approximation location close to a foot or at the foot and
a center of gravity approximation location at or close to the torso of the human or at or close to the center of gravity of the human
influences the detection of the connection information, and
particularly, the non-variable fixed portion of the relative position of the foot approximation location and an associated foot standing basis area (FBF1, FBF2) and/or between the center of gravity approximation location and the center of gravity influences the detection of the connection information.

10. Method according to claim 1, characterized in that the variable portion is determined by
using data from one or more sensors which measure the bending of a knee directly or indirectly,
using data from one or more sensors which measure the position of a hip joint directly or indirectly,
using the length of a lower leg and a thigh and
detecting a variable portion between the torso and the lower leg of the human,
wherein, particularly for indirect measurement, an inclination sensor for measurement of the inclination in relation to gravity and/or an acceleration sensor with a static measurement function is used which are, preferably, integrated into a garment, and/or, preferably, interfering accelerations from a rotation of a part of the body with which the inclination sensors or acceleration sensors are connected mechanically for measurement, are compensated for by help of an angular rate sensor and/or, for direct measurement, angle sensors are used which are, for measurement of an angle of parts of the body in relation to each other, connected with these parts of the body and which are, particularly, integrated into garment.

11. Method according to claim 1, characterized in that, as reaction on a risk of fall, a beginning fall and/or a ground fall, an alert is triggered in order to make the human attentive for the threatening fall or the fall, wherein the alert is particularly an acoustic and/or optical signal, a vibration which is transferred to the body of the human, a thermal and/or mechanical stimulus and/or an electrical stimulus of the human, for example by electrodes on the skin, and/or a chemical stimulus of the human, for example in the nose or on the skin.

12. Apparatus (100) for recognizing a risk of a tilt fall or a tilt fall, wherein the apparatus (100) is configured for carrying out of a method according to one or more of the preceding claims.

13. Fall projection garment (10) with an apparatus according to claim 12, wherein, particularly, the fall projection garment (10) has the form of an underwear (11, 12), wherein, particularly, the underwear (11, 12) is realized tightfitting at least at a location at which a sensor is arranged.
